(19) United States
(12) Patent Application Publication
(10) Pub. No.: US 2010/0113921 A1
(43) Pub. Date: May 6, 2010

(54) Systems and Methods for Object Surface Estimation

(75) Inventors: Elise FEAR, Calgary (CA); Jeff SILL, Calgary (CA); Trevor WILLIAMS, Calgary (CA); Martin KUHLMANN, Marsens (CH); Michael OKONIEWSKI, Calgary (CA); Jeremie BOURQUI, Calgary (CA)

Correspondence Address: FULBRIGHT & JAWORSKI L.L.P., 600 CONGRESS AVE., SUITE 2400, AUSTIN, TX, 78701, US

(73) Assignee: UTI LIMITED PARTNERSHIP

(21) Appl. No.: 12/476,957

(22) Filed: Jun. 2, 2009

Related U.S. Application Data
(60) Provisional application No. 61/058,135, filed on Jun. 2, 2008.

(51) Int. Cl. A61B 6/00; A61B 8/14

(52) U.S. Cl. 600/430; 600/443

(57) ABSTRACT

Systems and methods are provided that provide for surface estimation of an object. In particular, the surface estimation can be determined with little or no a priori information regarding the position or topography of the object within a given volume. In select embodiments, the systems and methods can be used for microwave imaging, and particularly for estimating breast surfaces during the imaging process.

### CROSS-REFERENCE TO RELATED APPLICATIONS

This application claims priority to U.S. Provisional Application No. 61/058,135 filed Jun. 2, 2008, the entire text of which disclosure is specifically incorporated by reference herein without disclaimer.

### BACKGROUND OF THE INVENTION

1. Field of the Invention

This disclosure relates to scan patterns, and more particularly to scan patterns for estimating unknown surfaces with reflection data.

2. Description of Related Art

Imaging, through various means, can generally allow visualization of objects that are obscured from the human eye. In medical applications, for example, imaging can allow a physician to probe internal parts of a body without resorting to invasive procedures such as open surgery. The choice of which imaging modality to use can depend upon the disease or trauma the physician wishes to visualize. For example, a broken arm may, in certain situations, warrant a simple x-ray to determine the extent of bone fracture. In other circumstances, however, magnetic resonance imaging may be justified to determine if a particular disease is present within bone.

In some cases, imaging can include transforming object signal data that corresponds to object structure into a form that is generally recognizable by some attribute, such as object shape. For example, MRI signal data may consist largely of tables of binary information that, when viewed, looks nothing like the object structure being interrogated. Computer algorithms and programs can transform this information into recognizable images that a physician can use to interpret disease.

### SUMMARY OF THE INVENTION

From the foregoing discussion, it should be apparent that a need exists for a system and method for object surface estimation.

An embodiment of a method for object surface estimation is presented. In one embodiment, the method includes receiving, at a sensor, one or more reflections from a surface of an object while the sensor is scanned in a pattern that localizes the surface of the object within a medium. The method may also include converting the reflections into one or more signals. Additionally, the method may include estimating a position of the surface of the object within the medium in response to the one or more signals.

In a further embodiment, the method may include locating a physiological feature on the object using a grid scan. The grid scan may include receiving signals corresponding to reflections from the object surface while the sensor is moved in a pre-defined path, and determining a distance from the sensor to the physiological feature. Additionally, the method may include performing a scan in a selected area of the surface of the object, wherein an antenna scan pattern is determined in response to information from a coordinate representation of the surface of the object. In another embodiment, the method may include generating a model of the surface of the object in response to a plurality of estimates of the position of a plurality of portions of the surface of the object within the medium.

In a further embodiment, the method may include conducting a first scan of the surface of the object with a first sensor, and conducting a second scan, substantially concurrently with the first scan, of an interior portion of the object with a second sensor. Additionally, the method may include positioning an antenna within a predetermined proximity of the surface of the object in response to the estimate of the position of the surface of the object within the medium.

In a certain embodiment, the method includes emitting a beam of light from a laser directed at the surface of the object, receiving, at a photo-detector, reflections of the light from the surface of the object, and converting the received reflections into one or more electrical signals. In an alternative embodiment, the method may include moving an antenna that receives microwave energy reflections from the surface of the object in a pre-selected pattern, the pre-selected pattern being defined to allow overlap of a first geometrically-estimated area calculated from said energy reflection on the object surface at a first antenna location with a second geometrically-estimated area calculated from the energy reflection on the object surface at a second antenna location, and generating a coordinate representation of the surface of the object. In still another embodiment, the method may include emitting an ultrasonic pulse from a transducer, the ultrasonic pulse directed at the surface of the object, receiving a reflection of the ultrasonic pulse from the surface of the object, and converting the received reflection into one or more electrical signals. Alternatively, the method may include capturing a digital image of the surface of the object with a digital camera, and estimating the location of the surface of the object in response to one or more properties of pixels comprising the digital image.

A system is also presented for object surface estimation. In one embodiment, the system includes a sensor. The sensor may receive one or more reflections from a surface of an object while being scanned in a pattern that localizes the surface of object within a medium, and convert the reflections into one or more signals. Additionally, the system may include a signal processing device coupled to the sensor, the signal processing device configured to estimate a position of the surface of the object within the medium in response to the one or more signals.

In a further embodiment, the signal processing device may locate a physiological feature on the object in response to data received from a grid scan. The grid scan may include receiving signals corresponding to reflections from the object surface while the sensor is moved in a pre-defined path, and determining a distance from the sensor to the physiological feature. In another embodiment, the sensor is further configured to scan in a selected area of the object, wherein a sensor scan pattern is determined in response to information from the coordinate representation or any other means of determining an outline of the surface of the object.

In another embodiment, the system includes a first sensor configured to conduct a first scan of the surface of the object, and a second sensor configured to conduct a second scan, substantially concurrently with the first scan, of an interior portion of the object. In a further embodiment, the signal processing device is further configured to generate a model of the surface of the object in response to a plurality of estimates of the position of a plurality of portions of the surface of the object within the medium. In a further embodiment, the system includes a positioning arm coupled to an antenna, the positioning arm configured to position the antenna within a predetermined proximity of the surface of the object in response to the estimate of the position of the surface of the object within the medium.

In one embodiment, the system includes a laser configured to emit a beam of light directed at the object, and a photo-detector configured to receive reflections of the light from the surface of the object, and convert the received reflections into one or more electrical signals. Alternatively, the system may include an antenna configured to receive microwave energy reflections from an object surface, and a positioning arm coupled to the antenna, the positioning arm configured to move the antenna in a pre-selected pattern, the pre-selected pattern being defined to allow overlap of a first geometrically-estimated area calculated from the energy reflection on the object surface at a first antenna location with a second geometrically-estimated area calculated from the energy reflection on the object surface at a second antenna location. In an alternative embodiment, the system may include an ultrasound transducer configured to emit an ultrasonic pulse directed at the surface of the object, receive a reflection of the ultrasonic pulse from the surface of the object, and convert the received reflection into one or more electrical signals. In still another embodiment, the system may include a digital camera configured to capture a digital image of the surface of the object, and the signal processing device coupled to the digital camera, the signal processing device configured to estimate the location of the surface of the object in response to one or more properties of pixels comprising the digital image.

The term “coupled” is defined as connected, although not necessarily directly, and not necessarily mechanically.

The terms “a” and “an” are defined as one or more unless this disclosure explicitly requires otherwise.

The term “substantially” and its variations are defined as being largely but not necessarily wholly what is specified as understood by one of ordinary skill in the art, and in one non-limiting embodiment “substantially” refers to ranges within 10%, preferably within 5%, more preferably within 1%, and most preferably within 0.5% of what is specified.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements possesses those one or more steps or elements, but is not limited to possessing only those one or more elements. Likewise, a step of a method or an element of a device that “comprises,” “has,” “includes” or “contains” one or more features possesses those one or more features, but is not limited to possessing only those one or more features. Furthermore, a device or structure that is configured in a certain way is configured in at least that way, but may also be configured in ways that are not listed.

Other features and associated advantages will become apparent with reference to the following detailed description of specific embodiments in connection with the accompanying drawings.

### BRIEF DESCRIPTION OF DRAWINGS

FIG. 1 shows an antenna path during a scan pattern according to one embodiment.

FIG. 2 shows charts of surface reconstruction according to one embodiment.

FIG. 3 is a hemispherical breast model, according to one embodiment.

FIG. 4 is a coronal outline of the model illustrated in FIG. 3.

FIG. 5 is a chart representing the breast model illustrated in FIG. 3.

FIG. 6 show illustrations of realistic breast model embodiments.

FIG. 7 show breast surface estimates created using an embodiment of a two-dimensional scan.

FIG. 8 illustrates a true surface for a breast model (a) and a corresponding estimated breast surface (b) according to one embodiment.

FIG. 9 illustrates a true surface (a) a corresponding estimated breast surface (b) for a breast model according to one embodiment.

FIG. 10 illustrates antenna positions for a grid scan pattern, according to one embodiment.

FIG. 11 illustrates locations on the breast closest to each antenna, according to one embodiment.

FIG. 12 is a chart illustrating distance to a breast model from antennas in selected rows of the cylindrical scan versus antenna number, according to one embodiment.

FIG. 13 illustrates locations on the breast closest to the depicted antennas in one row of the cylindrical or perimeter scan and two grid scan sizes, according to one embodiment.

FIG. 14 illustrates the error locations for an estimated breast surface on the simulated breast model, according to one embodiment.

FIG. 15 is a diagram illustrating error assessment used in one embodiment.

FIG. 16 is a flow diagram of an antenna placement algorithm according to one embodiment.

FIG. 17 is an illustration of a true surface for a breast model.

FIG. 18 is a chart of distance from antenna to skin vs. antenna number.

FIGS. 19A-19D illustrate an antenna positioning system, according to one embodiment.

FIG. 20 is a coordinate system that can be used in conjunction with an antenna positioning system, according to one embodiment.

FIG. 21 illustrates various movement definitions that can be used in conjunction with an antenna positioning system, according to one embodiment.

FIG. 22 is a schematic block diagram illustrating one embodiment of a top view of another embodiment of a system for object surface estimation.

FIG. 23A is a diagram illustrating one embodiment of a microwave scan pattern.

FIG. 23B is a diagram illustrating one embodiment of a laser scan pattern.

FIG. 24A is a diagram illustrating one embodiment of a microwave point estimate.

FIG. 24B is a diagram illustrating one embodiment of a laser point estimate.

FIG. 25A is a diagram illustrating one embodiment of a microwave surface reconstruction.

FIG. 25B is a diagram illustrating one embodiment of a laser surface reconstruction.

FIG. 26 is a photograph illustrating one embodiment of a laser sensor.

Like reference symbols in the various drawings indicate like elements.

### DETAILED DESCRIPTION

In general, analysis of biological structures using medical imaging methods can be enhanced using an accurate estimation of one or more of the surface topography, outline, and volume of the structure. In some cancer screening imaging modalities, for example, creating an estimate of the surface outline of the target organ or tissue can be an important factor in developing an overall imaging approach. Some radar-based microwave imaging techniques require an antenna to be placed a known distance from, or on the surface of, the object to be imaged. In some cases this may require a priori knowledge of the object location, size, and shape, which may be difficult to determine precisely in some circumstances. An imaging method providing the ability to scan an object using, e.g., lasers, mobile antennas, ultrasound transducers, or photographic equipment, without any prior knowledge of the object properties would be advantageous in a number of ways.

In general, methods for scanning a surface of a biological structure and estimating the location of the surface relative to the antenna locations are disclosed. One embodiment of the method is exemplified in the Tissue Sensing Adaptive Radar (TSAR) method (described below). Another embodiment of the method is described in the laser imaging method (described below). It will be understood that the methods are equally practicable in other scanning methods and analytical approaches whose objective it is to determine, for example, one or more of structure, volume, and surface features (including surface area) of an object. Additional examples may include ultrasound systems, photographic imaging systems, and the like.

Some imaging techniques use an antenna that directs energy toward a region of interest (such as a breast) and also receives scattered or reflected signals from the region, or from objects within the region. An example of such a technique is TSAR, described in U.S. patent application Ser. No. 10/942,945, which is incorporated herein by reference in its entirety.

Generally, TSAR is a low-power, ultra-wideband microwave imaging method that has been proposed for early stage breast cancer detection. Radar-based breast imaging can include illuminating the breast with short pulses of microwave energy and recording reflections. An image of the breast interior can be constructed in one embodiment by creating a synthetic array of antennas. In one embodiment, a synthetic array can be a single antenna placed at a number of locations, where a signal is collected at each point.

One operational theory of TSAR is that differences in the electromagnetic properties of healthy and diseased tissues can be expected to result in differences in the reflected signals. The reflected signals can be typically composed of a skin reflection and reflections from the breast interior, e.g., interfaces between fatty tissue, glandular tissue and tumors. The reflected signals can be analyzed and focused to create images that indicate the location of strongly scattering objects.

In some implementations of TSAR, an active ultra-wideband (UWB) monostatic radar approach is used to create an electromagnetic scattering map of the interior of a target imaging region, such as a breast. In some implementations of TSAR, a person may lie prone on a table, where a single breast can be suspended in a scan chamber through an opening in the table. The scan chamber can contain one or more antennas and an immersion medium into which the breast is immersed, either fully or partially. In another embodiment, the scan chamber may include a laser emitting device, a camera, an ultrasound transducer, or the like. Data relating to the breast and its surface, including topology, area, and other characteristics can be determined using antenna scan patterns and surface estimation algorithms, such as those disclosed below.

Preliminary scans, i.e., those that are performed to provide an imaging outline of the entire breast, may yield real coordinates of the skin surface with respect to the antenna; these data can provide information for subsequent scan patterns required for tumor detection. In one embodiment, a preliminary scan can be performed with a microwave antenna. In other embodiments, any modality that provides information for subsequent scans may be used, for example, photographic, optical (both laser or stereoscopic), acoustic, and mechanical methods of determining a surface outline may be used. The preliminary scan pattern can also aid in skin subtraction and focusing algorithms which can be used for tumor detection.

In general, a focusing algorithm can be enhanced using an estimated outline of the breast. For example, voxels included in the focusing algorithm may be limited to the voxels determined to exist inside the breast. This may lead to an increase in computational efficiency. In another example, signal propagation through different regions can be utilized in the focusing procedure. In one embodiment, time delays corresponding to the distances traveled in immersion medium and breast tissue can depend on both the properties of the materials and the distance traveled in each material. The surface estimate can be used to improve the estimates of distances traveled in each material relative to estimating the skin location at one point. Examples of this are discussed in U.S. patent application Ser. No. 12/407,671 which is incorporated herein by reference in its entirety. In some cases, for example using the skin estimation algorithm combined with a surface estimation algorithm, the outline of the inside of the skin can also be estimated. Signal propagation estimates may therefore be expanded to include propagation through the skin as well as the immersion medium and breast interior.

In some cases, a surface estimation algorithm can be used in an iterative manner. For example, a first estimated outline of a breast may indicate areas of low and high curvature where different concentrations of antenna or sensor locations may be appropriate. After additional data acquisition, a second and potentially more accurate outline may be created.

In some cases, parallel computing software and specialized hardware can be used to aid in signal processing speed.

In one embodiment, a priori knowledge of the breast location, shape, and size may be required for optimal antenna placement relative to the breast surface, and also to provide an accurate reconstruction of the processed image. In some cases this information may be necessary in order to prevent the antenna from contacting, or being placed at too great a distance from the breast during the imaging (i.e., scanning) process.

In general, generating an acceptable surface estimate of an object (e.g., a breast), can assist in the determination of certain variables for estimating the volume and other characteristics of the object. In some cases, a carefully chosen antenna scan pattern can have an effect on the generation of the surface outline.

The scan patterns described herein to irradiate the breast can be an important factor that may affect the outcome of the tumor detection efficacy, both in living samples and increasingly complex breast models used for research and development purposes. In some cases, the surface estimation process in TSAR uses no a priori information as to the shape or placement (within the scan region) of the breast, yet still operates successfully.

A second contribution generally includes accurately estimating the path lengths in the immersion medium, skin and breast interior. This estimation can benefit the skin subtraction, clutter reduction, focusing, and imaging steps in TSAR image reconstruction algorithms such as those disclosed in U.S. Patent Ser. No. 61/038,022 the contents of which are fully incorporated by reference herein.

In one embodiment of a scanning method, a first scan pattern may include a scan in a grid formation along the bottom of a scanning chamber, such as the scanning chamber described with respect to the TSAR method. The data in this grid scan may be used to approximate, among other things, the height of the breast nipple. A generic, multi-level cylindrical or coronal scan around the perimeter of the chamber can also be performed. The combined grid and perimeter scans can be used to estimate features relating to the surface of the breast, as described below. In one embodiment, the first scan may be carried out by a microwave antenna. Alternatively, the first scan may be carried out by a laser, an ultrasound transducer, a digital camera, or the like.

In one embodiment, the estimate of the breast surface obtained with the first scan may be used to identify appropriate antenna locations for the second scan. In one embodiment, the data from the second scan can be processed through a TSAR algorithm for tumor detection. Exemplary algorithms for processing TSAR data are disclosed in U.S. Patent Ser. No. 61/038,022, previously incorporated by reference.

In some cases, a method for breast surface estimation involves a detection scan in which an antenna or a laser may be scanned around the base and/or perimeter of the imaging region, and reflections of a short pulse can be recorded. The skin location can be estimated and this information can be used to create a simple signal that describes the location of the breast. The collection of simple signals obtained at the antenna and/or laser can be focused, and the resulting image can be processed to create a surface estimate.

In general, the methods provided herein for determining the surface of the breast may not require an a priori assumption regarding the breast position or shape in the array. In many respects, localization of the nipple is straightforward.

In general, a breast surface estimate may be used to define the region of interest for tumor detection, as well as in the design of imaging algorithms. In some implementations, the antennas, lasers or other sensors are scanned around the perimeter of the imaging region, resulting in separations between the antenna and breast of up to 11 cm. In this case, scanning the antenna closer to the breast may assist in increasing the tumor-to-clutter ratio in signals, as the illumination of the breast by the antenna can become more selective and the signal may experience less attenuation due to shorter propagation distances through the immersion medium. Therefore, in general, data from a surface estimate, conducted by various embodiments of a surface estimating technique or method, may be used to position the antennas at a desired distance from the breast for a second tumor detection scan. A system capable of antenna placement can be used to collect data for both the preliminary and subsequent scans. An antenna positioning system is generally described below that can be used for this purpose.

### Breast Surface Estimation

### Sensors

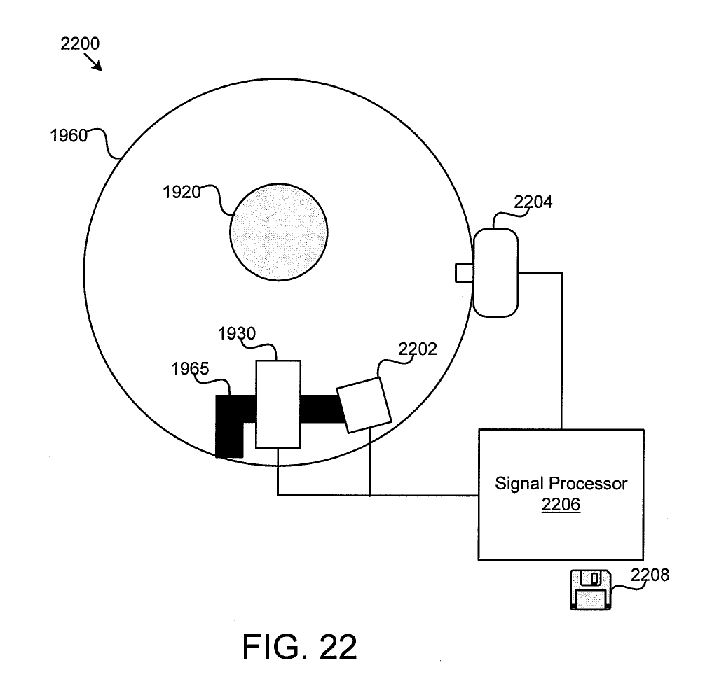

As described above, a breast surface estimation may include a first or preliminary scan of the breast. The first scan may collect information about the breast surface which may be used by a breast surface estimation algorithm to determine a model of the breast surface. The first scan may be conducted by a sensor. Various embodiments of a sensor are described below with reference to FIG. 22. In one embodiment, the sensor is the same microwave antenna used for the second scan. Alternatively, the sensor may include a laser, an ultrasound transducer, a digital cameral, or the like.

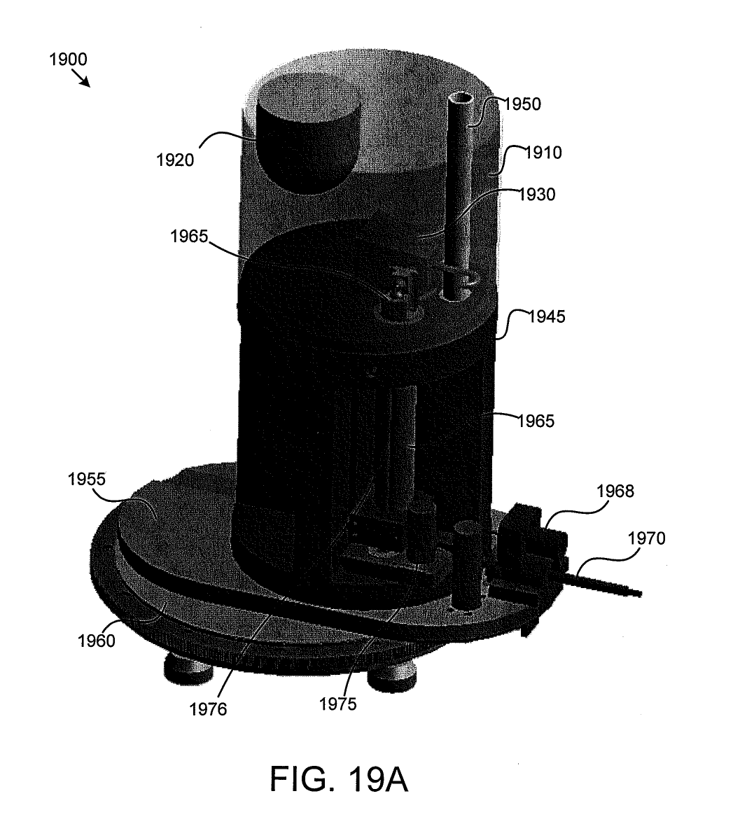

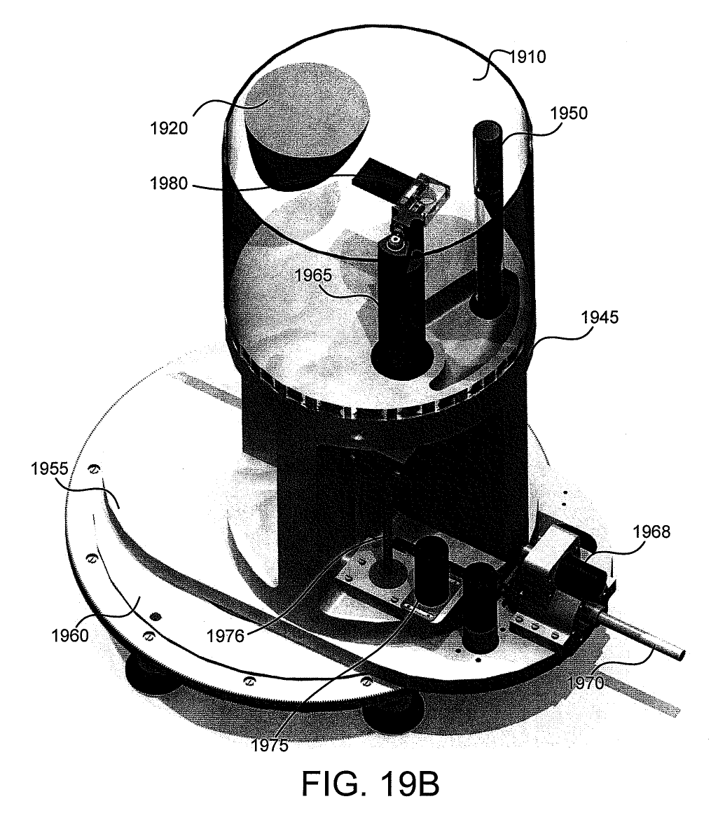

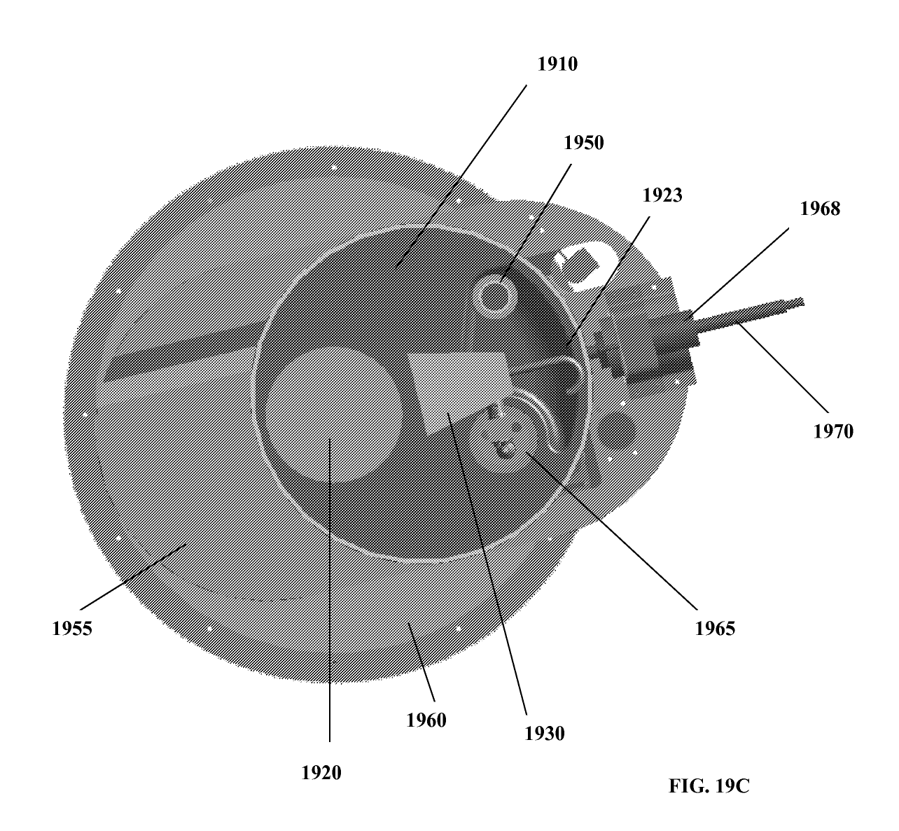

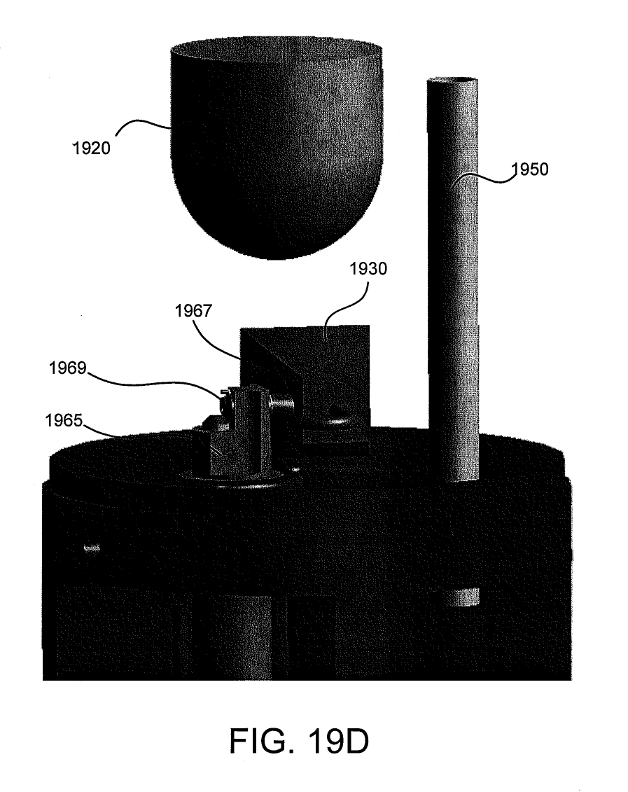

In one embodiment, the a microwave antenna sensor may include a Balanced Antipodal Vivaldi Antenna (“BAVA”) antenna. Alternatively, a BAVA-D configuration antenna may be used. In another embodiment, a Transverse ElectroMagnetic (“TEM”) horn antenna may be used. One embodiment of an antenna sensor is described below with reference to FIGS. 19A-B and FIG. 22. One of ordinary skill in the art may recognize alternative antenna configurations suitable for conducting the first scan. In one embodiment, the antenna may emit an Ultra Wide Band (“UWB”) beam or energy pulse. In a further embodiment, the antenna may be positioned such that the breast tissue is in the near-field region of the beam or energy pulse.

In an alternative embodiment, the sensor may be a laser. One embodiment of a laser is described below with reference to FIG. 22. By way of example, the laser may include a Reflex Laser Sensor adapted for measurement tasks available from e.g., Wenglor®. In a particular embodiment, Wenglor® model number CP24MHT80 may be adapted for use with the present embodiments. Of course, one of ordinary skill in the art will recognize that this example is merely for illustrative purposes, and that alternative laser sensor components available from various manufacturers may be used in accordance with the present embodiments.

One benefit of using a laser sensor is the small beam-width or footprint of the emitted light beam. This characteristic of a laser sensor may allow more precise tracking of the curvature of a breast. Another benefit may include in increased rate of sensing as compared with a microwave antenna. For example, the response time of some embodiments of a laser sensor may be between 50 μs and 50 ms, depending on the system configuration and the characteristics of the laser. This response time may greatly reduce the time required to perform the first scan when a laser sensor is implemented.

In a particular embodiment, the laser may be calibrated for operation in an oil medium. The calibration may be accomplished by scanning an a custom calibration piece, such as a cone or semi-sphere of known dimensions, from a known distance. In one embodiment the calibration may further including calculating medium dependent light propagation parameters based on a distance-voltage relationship. In one embodiment, the distance-voltage relationship may be expressed as d=aν+b, where d is the distance between the laser and the calibration piece, v is the voltage generated by the sensor, and a and b correspond to propagation parameters of the material through which the light propagates.

In an alternative embodiment, the first scan may be accomplished with an ultrasound transducer. The ultrasound transducer may include a piezoelectric transducer configured to generate a sonic pulse of a predetermined frequency in response to an electrical impulse. The sonic pulse may propagate through, e.g., the oil medium, reflect off of the breast surface, and receive a response pulse from the reflection of the breast surface. The response pulse may be converted into an electrical potential.

In another alternative embodiment, the first scan may be accomplished with a digital camera. One embodiment of a digital camera is described below with reference to FIG. 22. The digital camera may be configured to take video images. Alternatively, the digital camera may be configured to take still images. In such an embodiment, the estimation algorithm may utilize color, contrast, or other image specific data associated with pixels of the digital image to determine the contours of the breast surface. In light of the present embodiments, one of ordinary skill in the art of image processing will recognize methods for processing the digital images to estimate a breast model.

### Algorithm

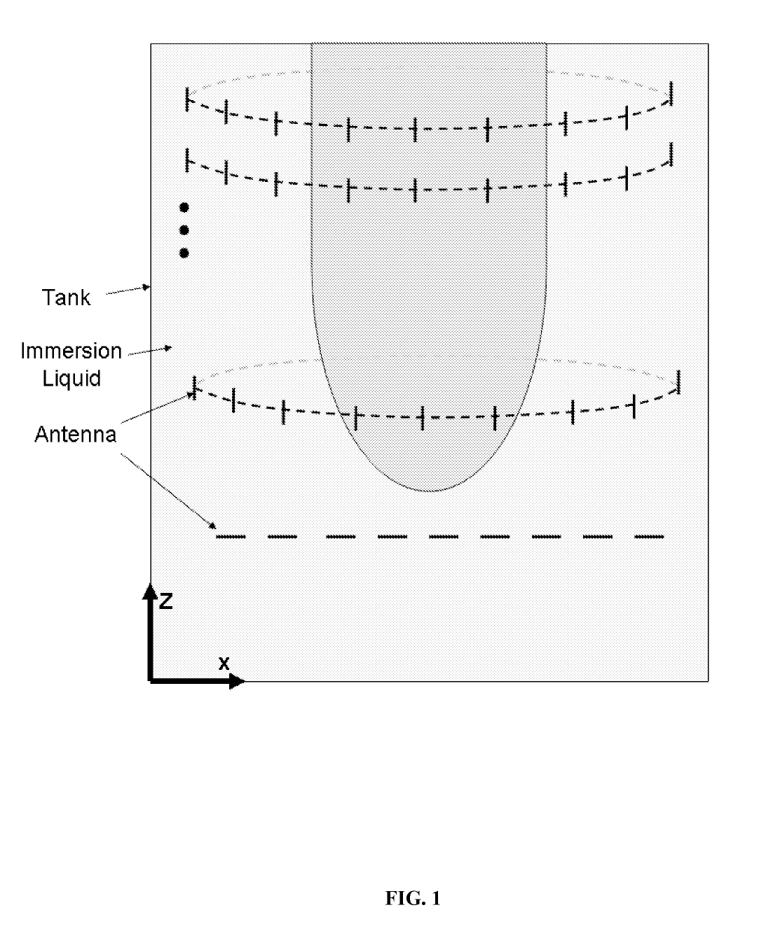

In one embodiment, the breast surface estimate can use a preliminary scan of the breast. This preliminary scan can be accomplished by moving the antenna (or multiple antennas) around the perimeter of the imaging region. A preliminary scan can include scanning the antenna(s) around the base and perimeter of the container, as illustrated in FIG. 1. In certain implementations, at each location, the antenna transmits and receives an ultra-wideband pulse of microwave energy which is captured as the raw imaging data.

A scan at the base of the container can include positioning the antenna parallel to the chest wall (the x direction in FIG. 1), and moving the antenna in intervals to create a planar synthetic array. The number of antenna scan locations can be determined by the approximate constraints of the container. For example, with a 30 cm diameter container, a grid scan that covers part of the base may be a 16 cm by 14 cm synthetic array with 72 scan locations created by moving the antenna in 2 cm increments. The data collected at these locations may be referred to as the base scan.

In general, the data collected during this portion of the scan can be used to determine the location of the nipple and create the surface outline of the lower portion of the breast, as described below. To collect further data with the perimeter scan, the antenna can be oriented in the z direction (FIG. 1) and moved through a set of circular scan paths at different z elevations.

The diameter of the circular scan paths may take into account certain variables such as the physical size of the tank, the antenna dimension. With an estimate of the location of the nipple and knowledge of the chest wall position (i.e. top of the tank), the extent of the perimeter scan in the z direction (FIG. 1) may be determined. In one embodiment, a resistively loaded dipole of length 2 cm may be used to collect data. With this specific antenna, the locations of the antenna feed point can range from slightly below the nipple to a minimum distance of 2 cm below the chest wall. This antenna/chest wall separation provides sufficient distance for the antenna to function properly. The number of rows, distance between rows, and number of antenna positions per row can be selected in order to provide an accurate, selected, or optimal surface estimate. In some cases, these variables can be varied in order to determine the effect of array parameters on the surface estimate.

In some cases, a breast surface estimate can be created by focusing a modified version of the recorded reflections, incorporating information and methods adapted from the TSAR algorithm. The skin location at each antenna can be estimated from the reflected signal using the impulse response technique. This method can include deconvolving the reflection from a reference object placed in the immersion liquid from the breast reflection. The skin location information can be used to create a simple signal from each reflection:

$${S}_{i} ( n ) = \{ \begin{matrix}0 , & n < {n}_{i} \\ 1 , & n \geq {n}_{i}\end{matrix} \qquad [ 1 ]$$

where Sᵢ is the signal corresponding to antenna i and nₗ is the time sample at which the skin location is estimated. The signals can be passed to the TSAR focusing algorithm, which essentially can time-shift and sum the data. At focal point location r, the pixel intensity can be calculated as:

$$E ( r ) = \frac{{\sum }_{i = 1}^{M} {S}_{i} ( 2 ( r - {r}_{i} ) / {v}_{ext} / dt )}{M} \qquad [ 2 ]$$

where rᵢ is the location of antenna i, νₑₓₜ is the estimate of the velocity in the immersion medium, dt is the time step and M is the number of antennas being used in the current surface estimation scan. When estimating the nipple location, M corresponds to the number of antennas in the base scan. When estimating the entire surface or volume of the breast, M can correspond to all antenna locations in the base and perimeter scans. To identify a surface from the resulting focused data, thresholding can be applied to E(r).

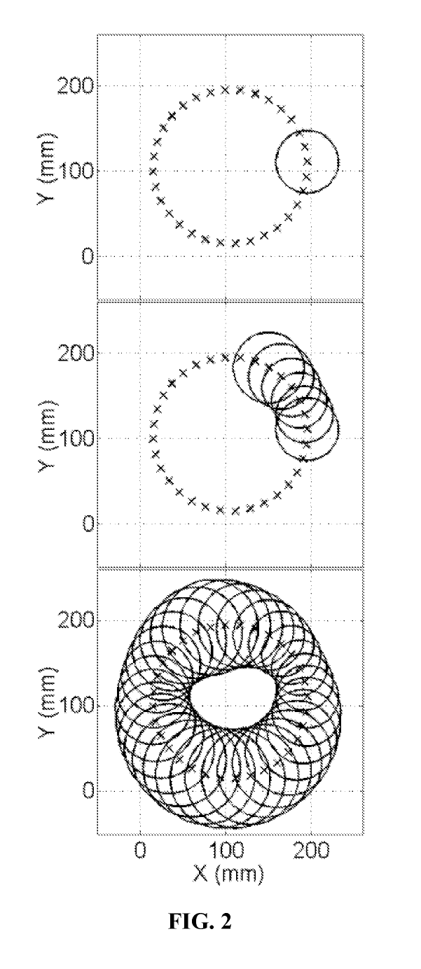

The threshold can be set such that pixels corresponding to the breast must contain contributions from all antennas; therefore, at each location r in the focused image, E(r) should equal one (1) for the point to be considered part of the breast. A physical interpretation of this criterion in two dimensions is shown in FIG. 2. Once an estimate of the skin location is found, a circle of corresponding radius with the antenna location at its center is created. The interior of the circle represents locations where the breast cannot exist, and, as FIG. 2 demonstrates, overlapping circles at neighboring antennas can create an outline of the breast estimate.

In some cases, two surface estimates can be created. Base scan data can be processed first, and the nipple location can be estimated from the result. This information can be used to design the perimeter scan, as described above. The algorithm can be applied to data recorded at all base and perimeter antennas, and the surface of the breast can be estimated from the nipple to the coronal plane of antennas closest to the chest wall. In most embodiments, extrapolation is not necessary to extend the surface artificially past the scan region. With a skin thickness estimate, the method described above may also be applied when determining the surface of the breast inside the skin. A second signal similar to equation (1) can be formed, where nₗ is simply modified to the time sample corresponding to the location and thickness of the skin layer. By taking the difference between the surface estimates created with the two signals, a representation of the skin layer can be obtained.

In some cases, multiple reflections may exist from the breast when the antenna is located such that, e.g., the chest wall and the breast are observed, i.e., detected. In some cases, determining the impulse response of the breast, i.e., using a deconvolution procedure to estimate skin location and thickness can assist in distinguishing the first and subsequent reflections for optimal use in TSAR imaging algorithms.

In one implementation, a deconvolution procedure can generally include the reflection from the object of interest, as well as a reflection from a known, calibration object, such as a metal plate. The impulse response, which describes the reflections from all material interfaces in the object of interest, can be extracted by deconvolving the reference signal from the reflection from the object of interest.

The algorithm described above is for estimation of the breast surface and calculation of a surface model in response to data received from a microwave antenna. In an alternative embodiment, a laser may be used to scan the breast surface. In such an embodiment, the algorithm described above may be adapted for use with laser data. Alternatively, the laser may be used in conjunction with commercially available surface estimation software to determine a breast model. For example, Powercrust™ software available from the University of Texas Department of Computer Science may be incorporated into, e.g., a dedicated signal processing device, for estimation of the breast model in response to data from the laser sensor.

### Examples

### Hemispherical Breast Model

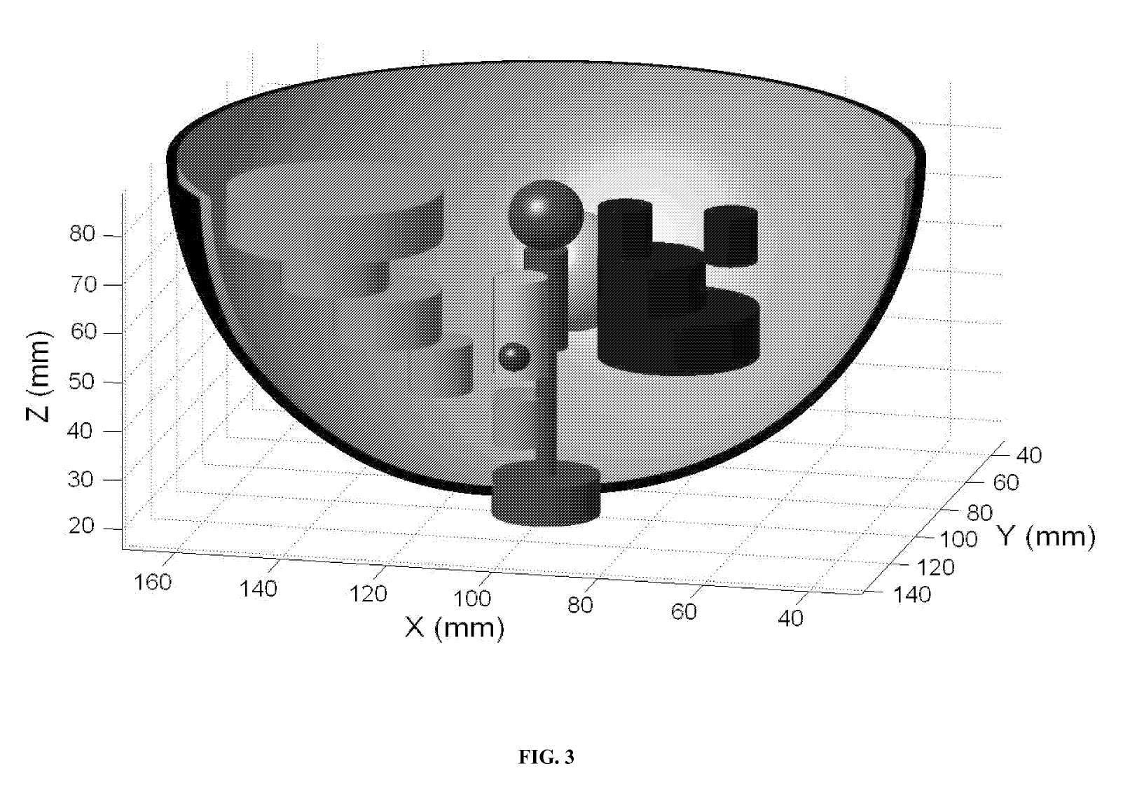

In this example, data were generated with the Finite Difference Time Domain (FDTD) software similar to previous work with TSAR. The hemispherical model is shown in FIG. 3. The “skin” of the 14-cm diameter hemisphere was uniformly 2.0 mm thick with a dielectric constant (εᵣ) of 36.0 and conductivity (σ) of 4.0 S/m. The hemisphere contained fat with electrical properties εᵣ=9.0, σ=0.4 S/m, and multiple glands with properties εᵣ=11.0 to εᵣ=15.0 and σ=0.4 to σ=0.5 S/m. The hemisphere contained a spherical tumor with εᵣ=50.0, σ=4.0 S/m, with center at location x=12.5 cm, y=10.0 cm and z=5.5 cm of diameter 6.0 mm. A cylindrical nipple of 2.0 cm diameter and properties εᵣ=45.0 and σ=5.0 S/m was also included. The nipple was placed from z=1.6 to z=2.4 cm, therefore extending out from the hemisphere by 4.0 mm. The hemisphere was immersed in fat with similar properties to the interior fat. The antenna was a Wu-King resistively loaded dipole with center frequency of 4.0 GHz and excited with a differentiated Gaussian pulse with approximately full width half maximum frequency range of 6 GHz.

Three simulation scenarios were performed. The first consisted of scanning the dipole in a circular path around the breast at z=5.5 cm (for orientation, refer to FIG. 3). Reflections were recorded at 20 antenna locations, each location separated by 18 degrees. The dipole was positioned vertically in the z direction and spanned from z=4.875 to z=6.125 cm. The center of the dipole was located 1.13 cm from the hemisphere, corresponding to minimum and maximum antenna distances of 0.93 cm to 1.39 cm, respectively.

The second simulation scenario consisted of scanning the antenna around the hemisphere in 9 circular paths with 10 antenna locations per row. A “path,” as used herein, implies a trajectory of an antenna on a two-dimensional plane of a coordinate system and a “row,” as used herein, implies the set of locations along a given path where measurements are performed. Alternate rows began at references corresponding to 0 and 18 degrees, and the antenna was moved in 36-degree increments until 10 antenna positions per row were acquired. The scans were performed from z=2.5 to z=6.5 cm in increments of 0.5 cm with the antennas aligned with the z direction.

The third scenario included creating an outline in the sagital plane. The antenna was positioned at x=10.0 cm (refer to FIG. 3) and scanned from z=2.0-7.0 cm in 0.5 cm increments. This was repeated for 2 antennas per row with 180-degree separation. A row of horizontally positioned antennas were scanned at z=0.6 cm, x=10.0 cm, from y=6.5-13.5 cm in 0.5 cm increments. Therefore, 37 antenna locations were used to create the sagital outline.

Information from every antenna location was used to create the outline of the hemisphere using the TSAR algorithm. First, skin location and thickness estimates are found using the impulse response method. The skin location and thickness is estimated for each antenna in the 9×10 array. The average antenna skin location and thickness estimates for scenario one are 1.16 cm and 1.75 mm respectively. This location estimate corresponds to a 2.57% error when compared to the center of the dipole. Next, a focusing program examined every voxel inside the imaging region to determine whether it was at a sufficient distance to be classified as outside or inside the model. Antennas from multiple rows and locations can have an influence in determining whether a specific voxel is classified as immersion medium, skin, or model interior in this method, and does not require any a priori knowledge about the shape of the model.

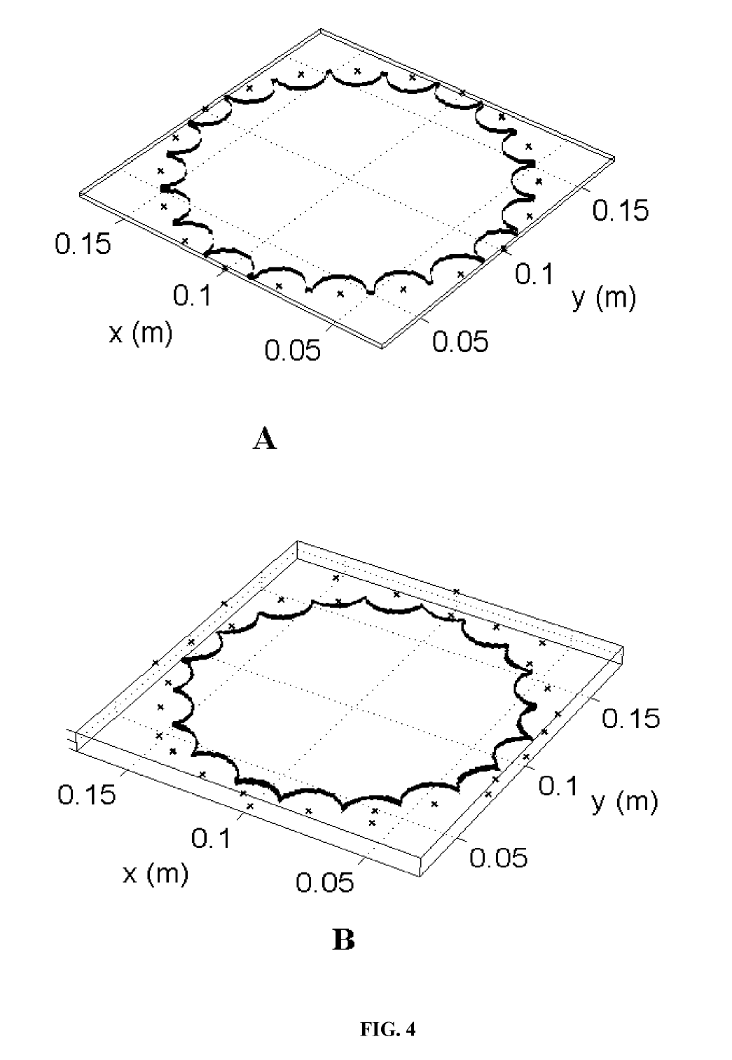

FIG. 4A shows the outline generated in the coronal plane for scenario one, where the small “x” indicators represent antenna locations. With 18 degrees of separation between each antenna, the outline corresponds well to the physical hemisphere where the line between the antenna and model center crosses the outline. However, midway between the antenna locations, the outline is constrained by the scan path, corresponding to an outline detection error distance of 1.13 cm. For comparison, a coronal outline at z=5.5 cm was created using the information from 3 rows of 10 antennas from z=5.0-6.0 cm. This is shown in FIG. 4B. The outline is not only constrained by the antenna scan path, as in scenario one. The maximum deviation from the true outline is 8.2 mm; a 27.5% improvement when compared to scenario one. From these data it may be considered that including multiple rows of antennas in outline creation is an important consideration.

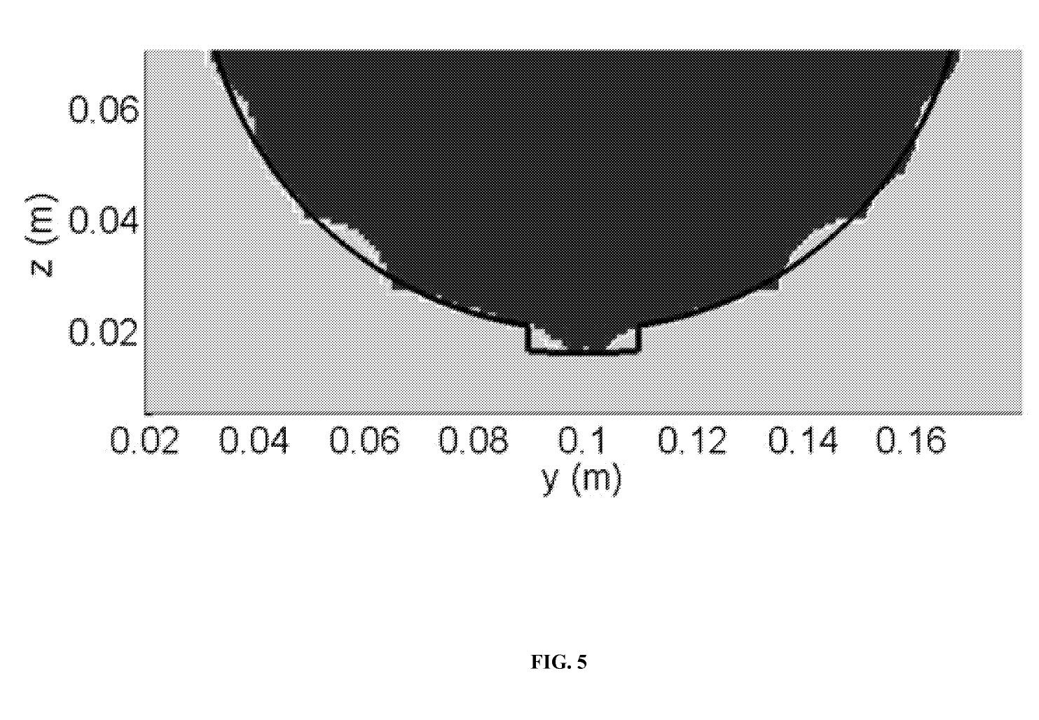

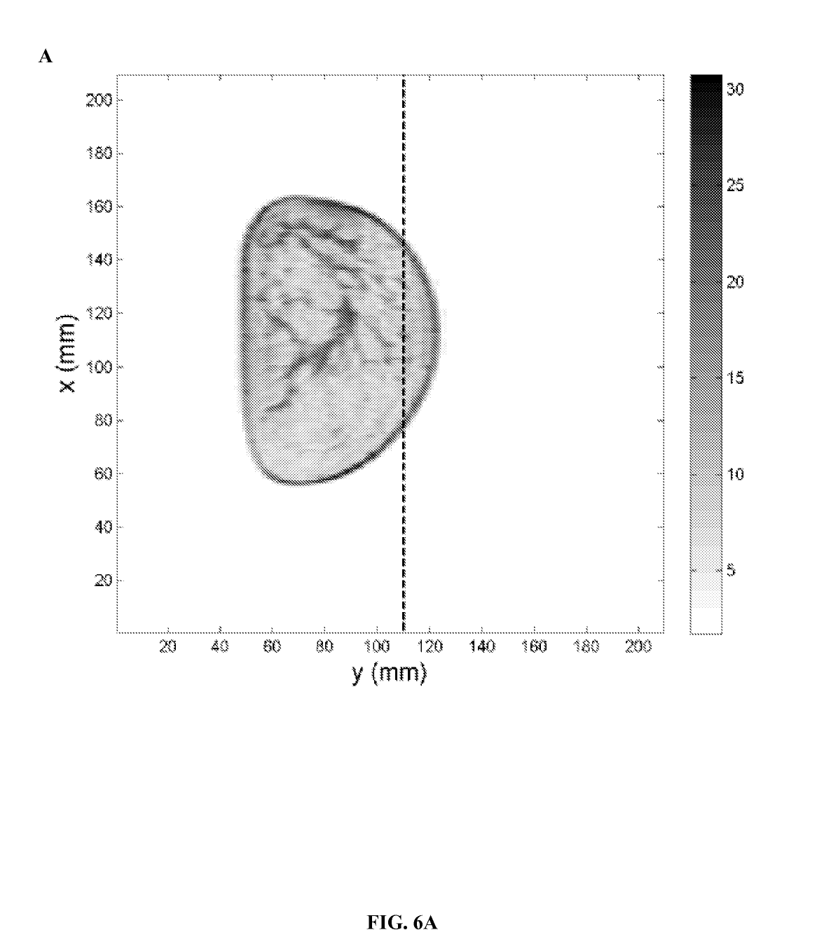

The sagital outline was generated using the antennas described as in scenario three, and is shown in FIG. 5. The outline represents the curvature of the hemisphere acceptably with a maximum deviation from the true hemisphere of 4.5 mm. The deviations evident in the outline on either side of the hemisphere at z=3.0 cm can indicate the importance of devising a scan pattern to adapt to specific regions of the breast. A maximum deviation from the nipple of 4.1 mm is evident at the edges. However, the nipple is visible and is estimated very well in its central region.

### Realistic Breast Models

More realistic models may be used to test the breast surface detection and antenna placement algorithms. These models may be derived from Magnetic Resonance (MR) images. The MR images may be converted into models suitable for use with an electromagnetics simulator. In this example, several complex models are created, however similar procedures are employed. Images are segmented by applying a threshold in order to identify the interior and exterior regions of the breast. The threshold is adjusted manually for each breast model to identify the interior of the breast, including the skin.

Breast model 1 is a simplified model consisting of a 2-mm thick layer of skin bounding a non-dispersive, homogeneous fatty tissue with εᵣ=9 and σ=0.4 S/m. The layer of skin with εᵣ=36 and σ=4 S/m is added at the boundary between the interior and exterior of the model that is identified in the thresholding step.

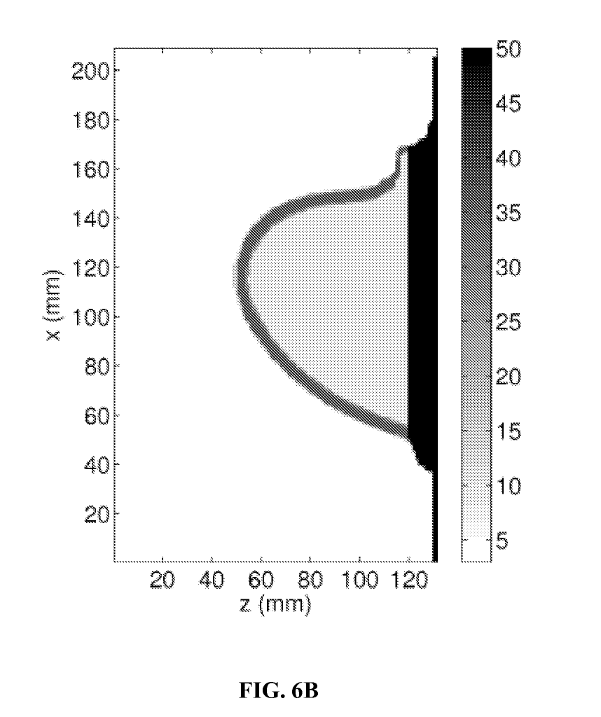

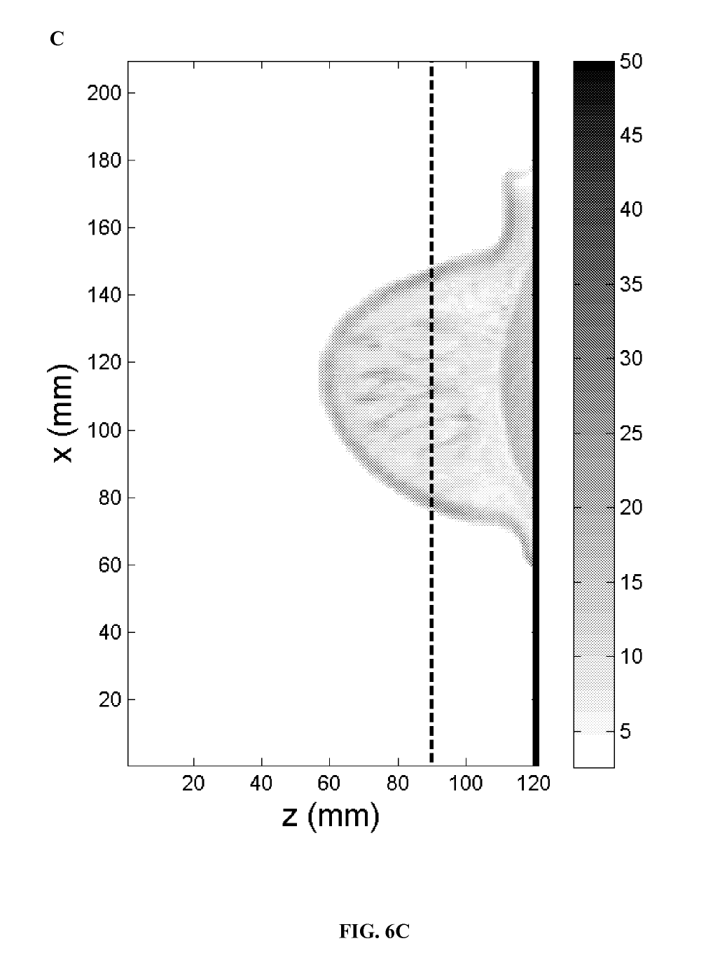

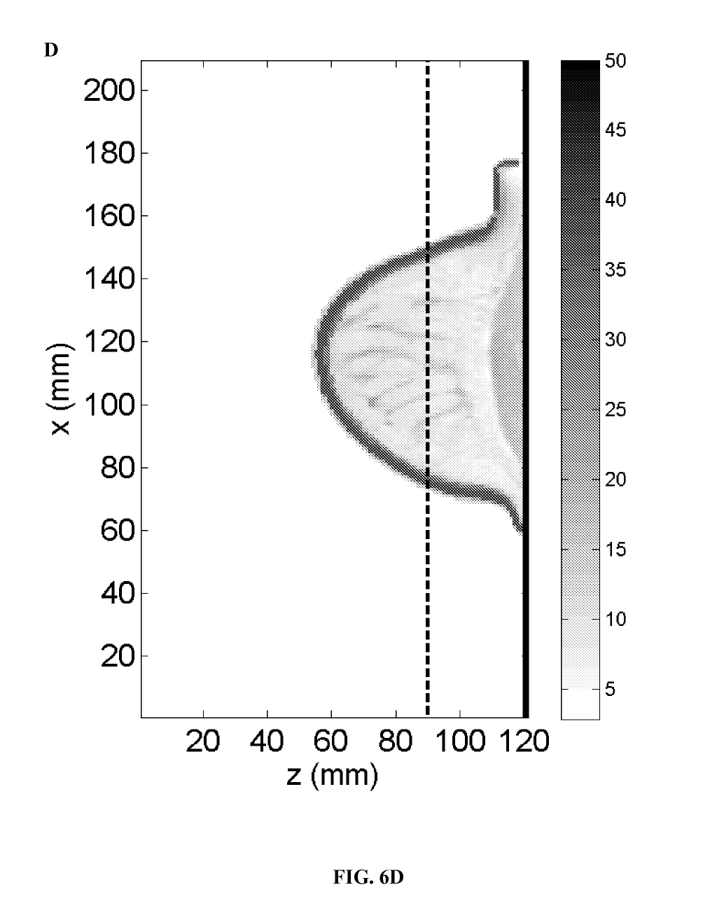

In breast model 2, variations in the MR pixel intensity are used to create variations in the electrical property distribution in the breast. The pixels of the breast are linearly mapped to the electrical properties by selecting the maximum required permittivity and conductivity values (εᵣ=36 and σ=4 S/m). A modified version of breast model 2 (2B) has constant properties assigned to the skin (εᵣ=36 and σ=4 S/m), which are identified in the MR image using pixel intensities. Breast model 2, therefore, has thickness and property variations in the skin, while the skin on breast model 2B exhibits thickness changes but maintains a constant electrical property of εᵣ=36 and σ=4 S/m. To increase the realistic nature of the model, a layer of muscle (εᵣ=50 and σ=4 S/m), representing the chest wall, is attached to the breast models. The breast models are placed in a lossless immersion liquid with similar electrical properties to canola oil (εᵣ=3.0). To illustrate the three models, FIG. 6 shows 2D slices through the 3D breast volumes. The tissues are modeled as non-dispersive, however this simple model is acceptable, as the initial skin reflection is the signal of interest.

Reflections from the breast model are obtained with simulations performed using the finite difference time domain (FDTD) method. A single, resistively loaded dipole antenna of length 2 cm transmits an ultra-wideband pulse and reflections are recorded at the same antenna. The transmitted pulse is a differentiated Gaussian signal with frequency content from 1-10 GHz. The excitation of the antenna with a pulse is simulated as the antenna is scanned through locations forming grid and perimeter scans, as described below.

For each antenna location, skin location is estimated first. For breast model 1, the actual distance between the antenna feed point and the closest point on the realistic model is calculated and found to track very closely with the estimated antenna/breast separations using the IR method, with a mean error of 1.50 mm over 224 (lower 5 of 7 scanned rows×32 positions) cylindrically scanned antennas from z=30 to z=70 mm. FIG. 6 indicates orientation of the models and the z axis.

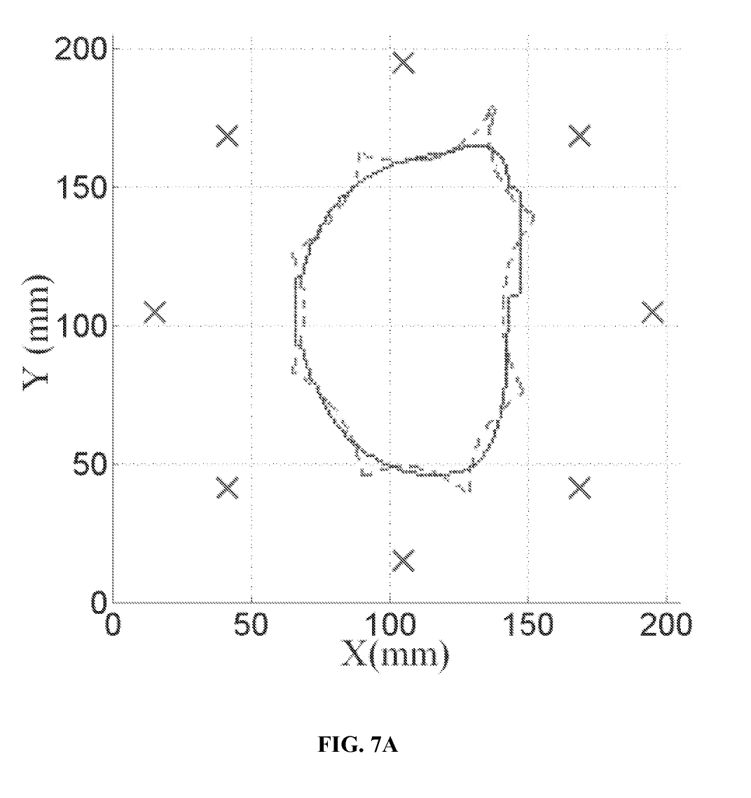

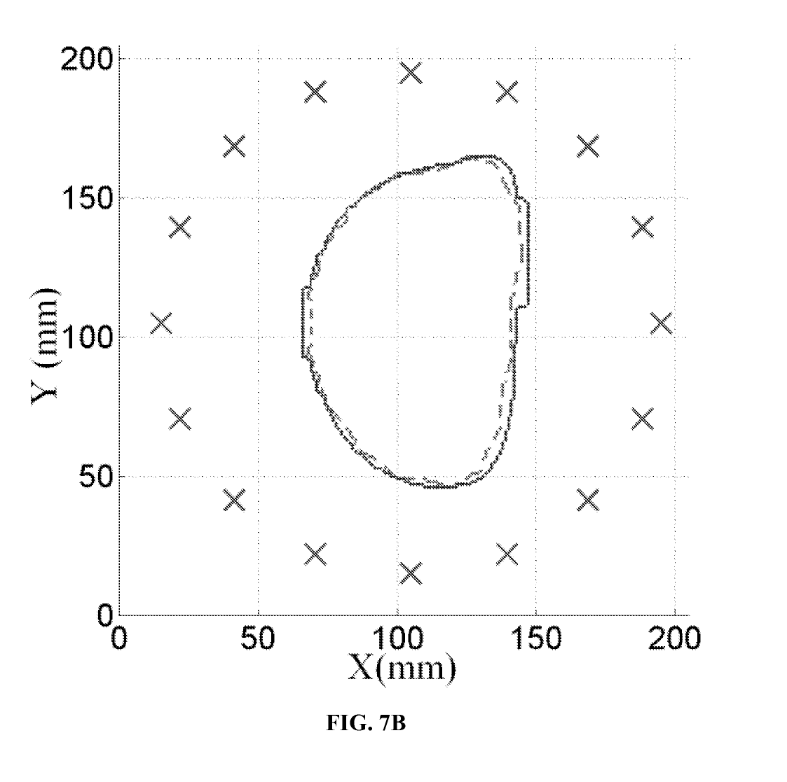

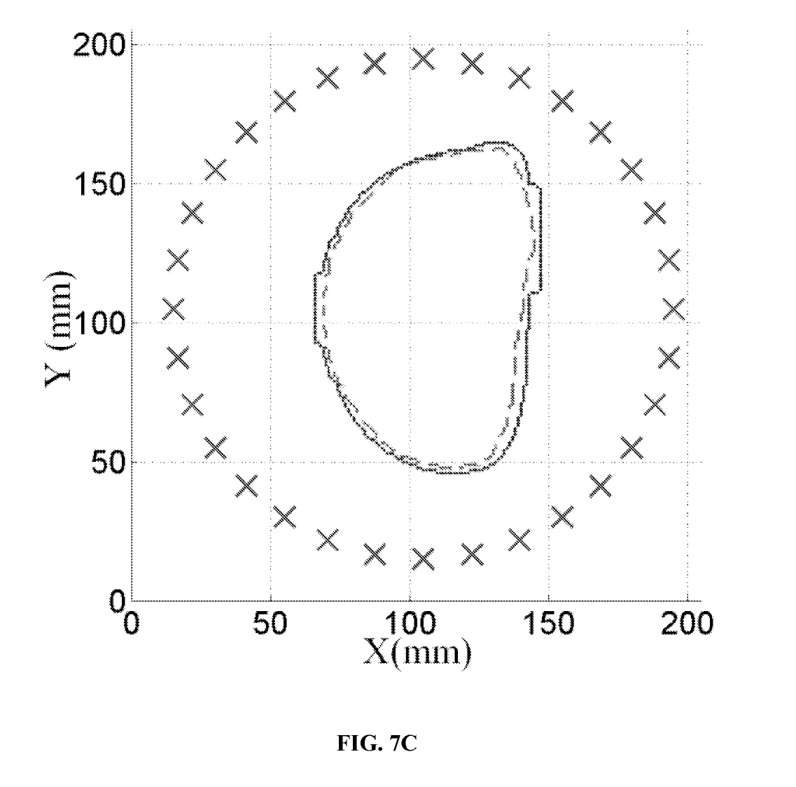

Next, surface estimates are constructed for the breast models using the algorithm described above. The performance of the surface estimation algorithm can be assessed by first considering a two-dimensional result. To understand the influence of the number of antennas per row, a 2D surface estimate can be created using breast model 1 and one row of the perimeter scan collected at z=103 mm and with path diameter of 18.0 cm. Results obtained using 8, 16, and 32 antennas are compared in FIG. 7. The actual skin location (solid line) is also included, and antenna locations are identified with an “x.” The surface estimate with 8 antennas provides a rough representation of the breast surface as shown in FIG. 7A, with maximum overestimation of 4.12 mm, maximum underestimation of 4.00 mm, and average errors of 2.27 mm and 1.70 mm, respectively. Error calculation methods are described below. FIG. 7A and FIG. 7B indicate that increasing the number of antennas can increase the smoothness, and thus accuracy, of the breast surface estimate. With 16 and 32 antennas, there is no overestimation, while the average errors are 1.70 mm and 1.77 mm, respectively. The antennas are an average distance of 4.45 cm away from the breast; at this location, the half-energy beamwidth of the resistively loaded dipole antenna is 6.7 cm.

Considering the scan diameter of 18 cm and 8 antennas, the beamwidths of neighboring antennas do not overlap significantly, giving rise to the errors observed in FIG. 7A. With 16 antennas, the beamwidths overlap significantly, and the error was greatly reduced. The increased overlap with 32 antennas did not significantly change the error in the 2D slice. This implies that the scan pattern should be designed such that sufficient overlap between the beamwidths of neighboring antennas is achieved.

Next, a full scan of breast model 1 was performed. The base scan was performed, and the resulting location of the nipple was estimated to be z=3.4 cm, while the actual location was 3.8 cm. Although there was a 4 mm error, the location was an estimate used to determine the number of rows for the perimeter scan.

The distance between the nipple estimate and chest wall was 9.7 cm. A row separation of 2 cm was selected, which corresponded to the length of the resistively loaded dipole. This resulted in 4 rows of antennas in the perimeter scan with antenna feed points spanning from z=40 mm to z=100 mm. Based on the results of the 2D estimate, 16 antennas per row were included. With multiple rows, increased information was provided, especially with judicious selection of scan pattern. Each row was rotationally offset by 7.5°, so every third row had the same antenna positioning. This provided offset antenna locations in rows above and below a selected row, while maintaining overlap between the antenna patterns.

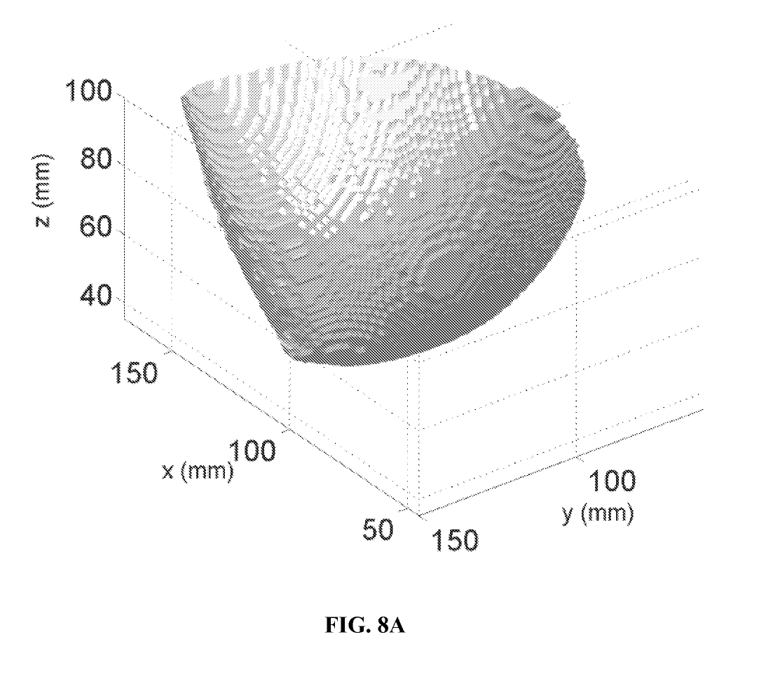

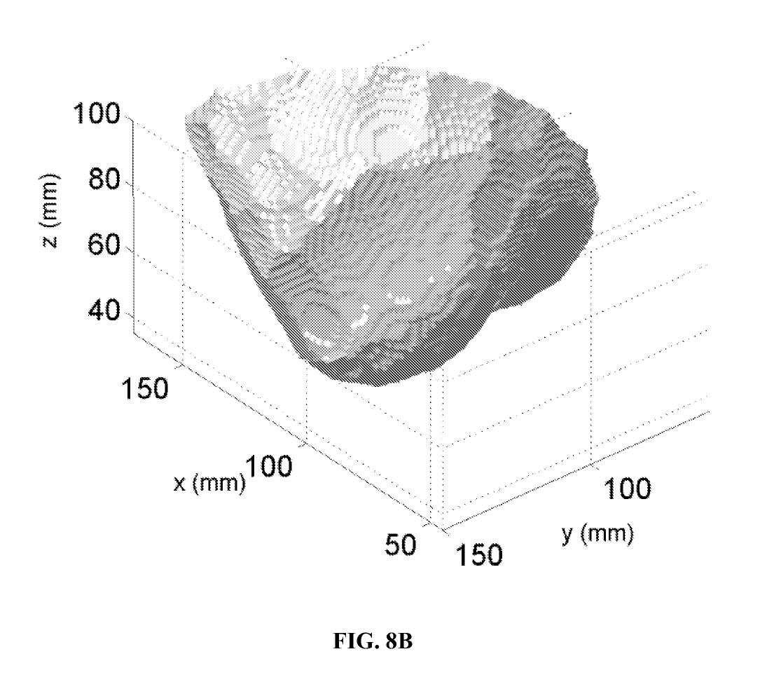

FIG. 8 shows the three-dimensional breast surface estimate created with 4 rows of 16 antennas and the base scan. The breast volume was slightly overestimated near the chest wall and nipple, and underestimated in the regions in-between. To quantitatively analyze the breast surface estimate, the maximum and average distance errors were computed for both over- and underestimation (Table 1). For both errors, the small average errors demonstrate the success of the algorithm. The maximum underestimation error occurred in the region of z=70 mm and x=80 mm, and may have been due to an overestimate of the skin location (relative to the antenna location that is computed) at an antenna in the base scan. The maximum overestimation occurred near the nipple, as the base scan may not have been dense enough to accommodate the complexity of the model in that region.

Another approach to assessing error is calculating the quantity of voxels in error. Taking into account the inherent slight overestimation of the skin location (relative to the antenna location) associated with the impulse response method, the larger number of underestimation error sites compared to overestimation was predictable. Although the number of error sites associated with underestimation was large, the average error value was low. Therefore, the error associated with most sites was expected to be 1 mm.

With breast model 1, the effect of an increased number of antennas was investigated. Breast surface estimates were created with varying numbers of rows and antennas per row, and errors are summarized in Table 1. First, results obtained with 4 and 9 rows are compared. The additional 5 rows were included in such a way that the array spanned from 30 mm to 110 mm in the z-direction with 1 cm separation between rows. For comparison purposes, the surface was first estimated to z=100 mm. The breast surface estimate was improved with the additional rows, especially near the chest wall. This was demonstrated by the decrease in maximum overestimation error, average overestimation error, and error sites. Next, an additional 16 antennas were added to each row of the 9×16 array. In this 9×32 array, scan locations were simply placed at the midpoint between the existing locations. This maintains a rotational offset that repeats every third row, however now the offset is 3.75°. The overestimation error did not change significantly with the added antennas, consistent with the observations in the 2D case. A slight improvement in underestimation error was noted. This again underscores the importance of a sufficient antenna scan pattern that minimizes data collection time while maximizing breast coverage by the antennas.

In all cases reported in Table 1 for breast model 1, the constant maximum underestimation error was attributed to the same base scan antenna. Enabling one antenna to have such a dominant affect on the error is undesirable. In some embodiments, understanding the effect of the threshold applied to E(r) on overall error may be an important consideration. Referring to FIG. 6B, a curvature is seen in the breast near the chest wall at approximately z=110 mm and x=160 mm. This appears to be a location with potential for large error due to the complexity of the model in this region. To understand how the scan pattern and omnidirectional antennas react to this region, the surface estimate was extended to z=110 mm. An increase in the maximum overestimation error for the 9×16 scan was observed, as the antennas detect the convex edge of the complexity. Combined with the method of surface detection, this resulted in a smoothing of the concave corner and finally an overestimation of the surface. With a 9×32 array, the maximum error remained at 2.00 mm and the average error increased slightly when compared to the estimate limited to z=100 mm. This was attributed to the combination of the slight overestimation of skin location and increased number of antennas, and suggests that an increased number of antennas may be beneficial when estimating surfaces with more complex contours.

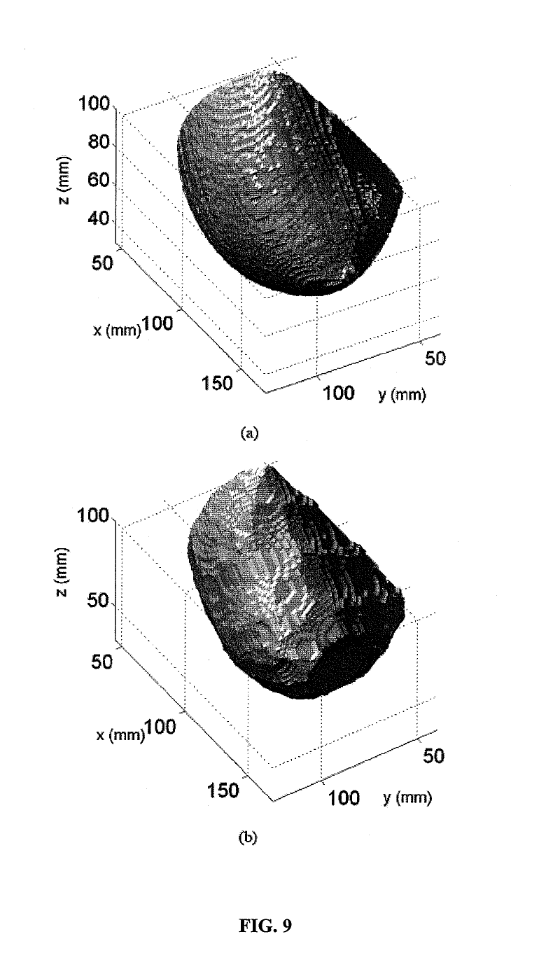

Surface estimates were created for breast models 2 and 2B. The base scan gave an estimate of the nipple location at z=3.9 cm, compared with the actual location of z=3.4 cm. The rows of antennas, therefore, started at z=4.0 cm and continued to 2 cm below the chest wall. With 1 cm separation between rows, this corresponded to a perimeter scan consisting of 7 rows with 16 antennas per row. Each path has diameter of 19.0 cm. The surface estimate for model 2 is shown in FIG. 9, and errors for models 2 and 2B are summarized in Table 1.

The small average errors obtained with models 2 and 2B demonstrate the success of the method and show that the algorithm is robust to variations in skin thickness and properties. Further, the size of this model and its placement inside the perimeter scan can ensure a more difficult surface to estimate. The model was slightly smaller than breast model 1, the perimeter scan was 1 cm greater in diameter, and the model was located in an offset position relative to the center of the scan pattern. Several of the perimeter antennas furthest away from the model are located significantly closer to the chest wall, so the skin location estimates at these antennas correspond to the chest wall interface. However, several antennas in the outlying regions of the base scan overestimated the skin location relative to the antenna. In combination, these errors in skin location resulted in surface estimates with no overestimation errors and increased underestimation errors when compared to breast model 1. The maximum errors obtained with both models corresponded to the region in which the perimeter scan antennas are located furthest from the model.

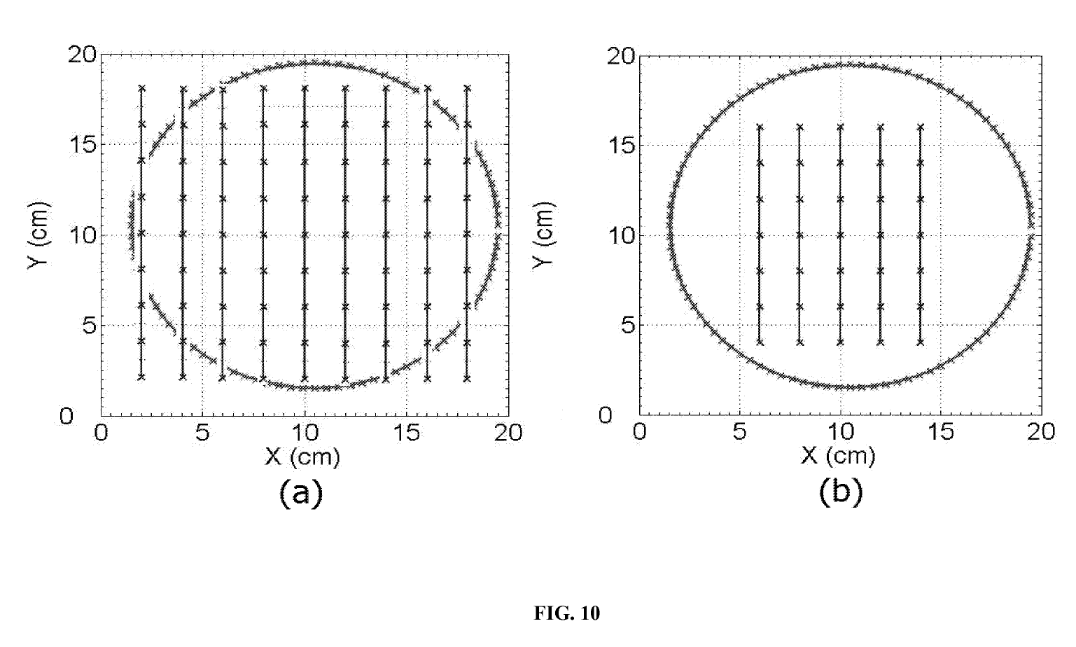

The results so far have demonstrated the effectiveness of the surface estimation algorithm. Next, the impact of antenna scan pattern, specifically the grid scan, on results is further explored. With breast model 1, two rows in the perimeter scan are selected. These rows contain 32 equally spaced positions on the circumference of the circle and are located at z=30 mm and z=40 mm. The grid antennas, placed perpendicular to the coronally scanned antennas, were configured in 9×9 and 5×7 arrays as shown in FIG. 10.

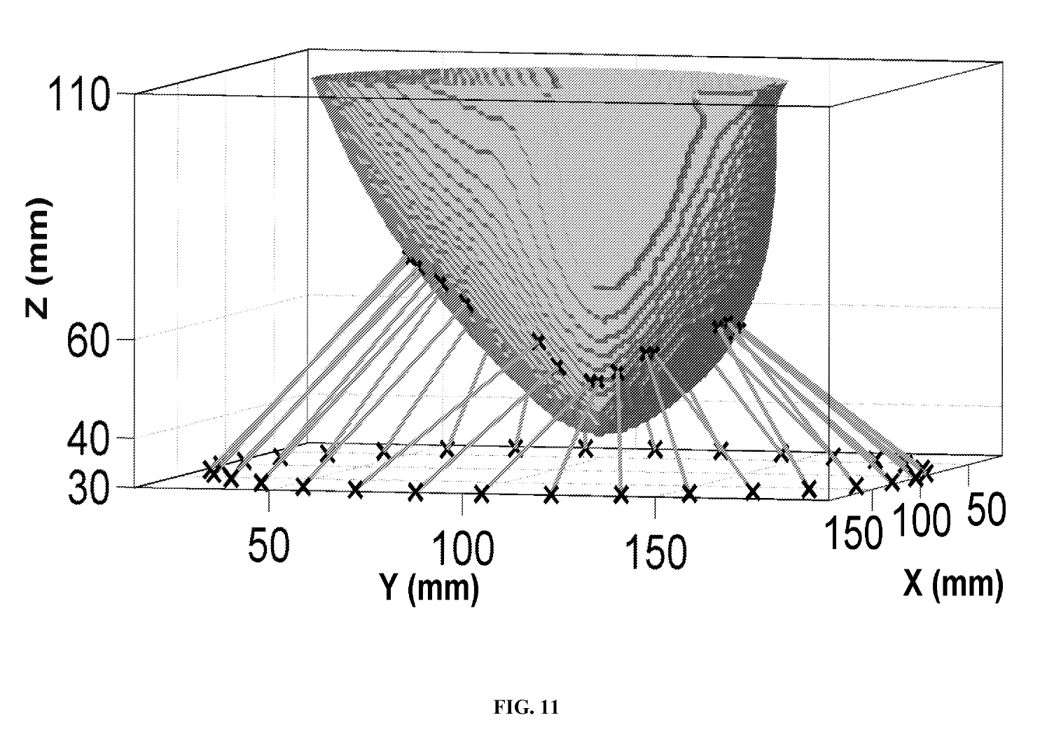

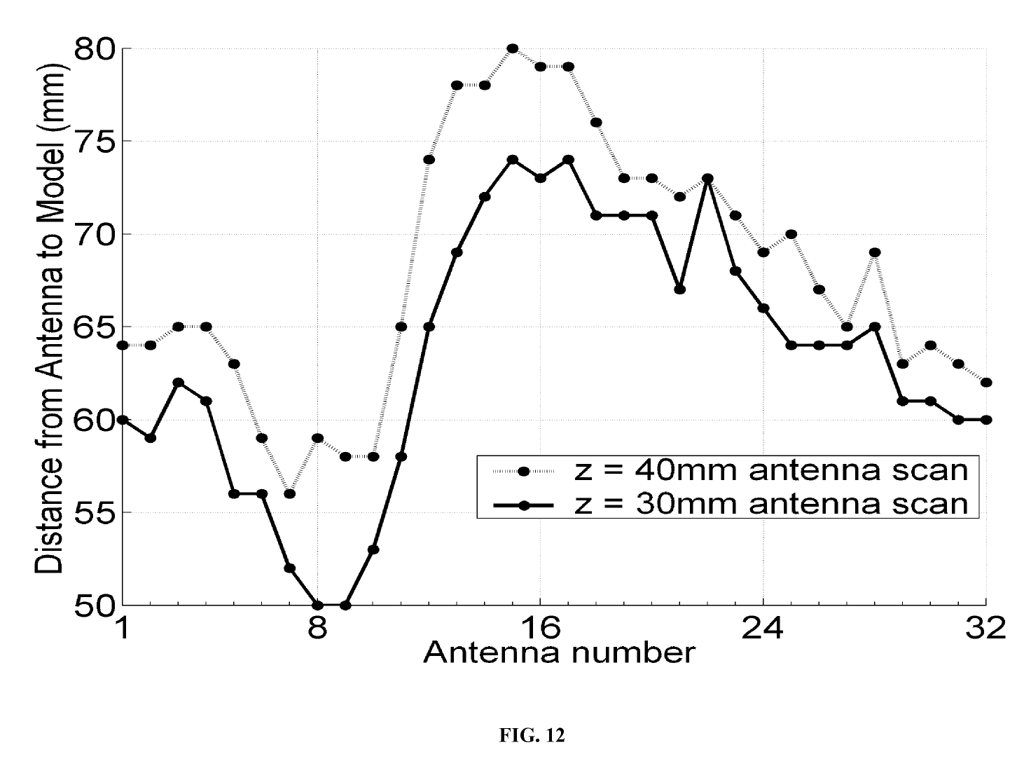

FIG. 11 shows one row and the closest location on the breast model relative to each antenna, specifically the row located at z=30 mm where the nipple begins at approximately z=40 mm. FIG. 12 illustrates that, for the z=30 mm scan pattern, the z-locations of the breast model ranges from approximately 50 mm to slightly less than 75 mm. For the antenna scan where z=40 mm, the z-locations are between 56 mm and 80 mm. For both rows, a z-location variation is observed. In other words, the closest distance between the antenna and breast model varies a great deal over one row of observations. For this model, there are no z-location values close to the nipple at approximately z=40 mm. FIG. 12 shows that not only is there a variation in the closest z-location to the breast model for both rows, but that due to the unsymmetrical (realistic) nature of the model, the variations are not consistent between rows.

The above observations suggest that the grid scans can be an important factor in estimating surface outline regions not illuminated by the perimeter scan (e.g., the nipple region). In one implementation, grid layer scans can be used when the lower perimeter rows do not illuminate the nipple sufficiently (e.g., as illustrated in FIG. 11). This may not be necessary when the system is implemented with a laser sensor.

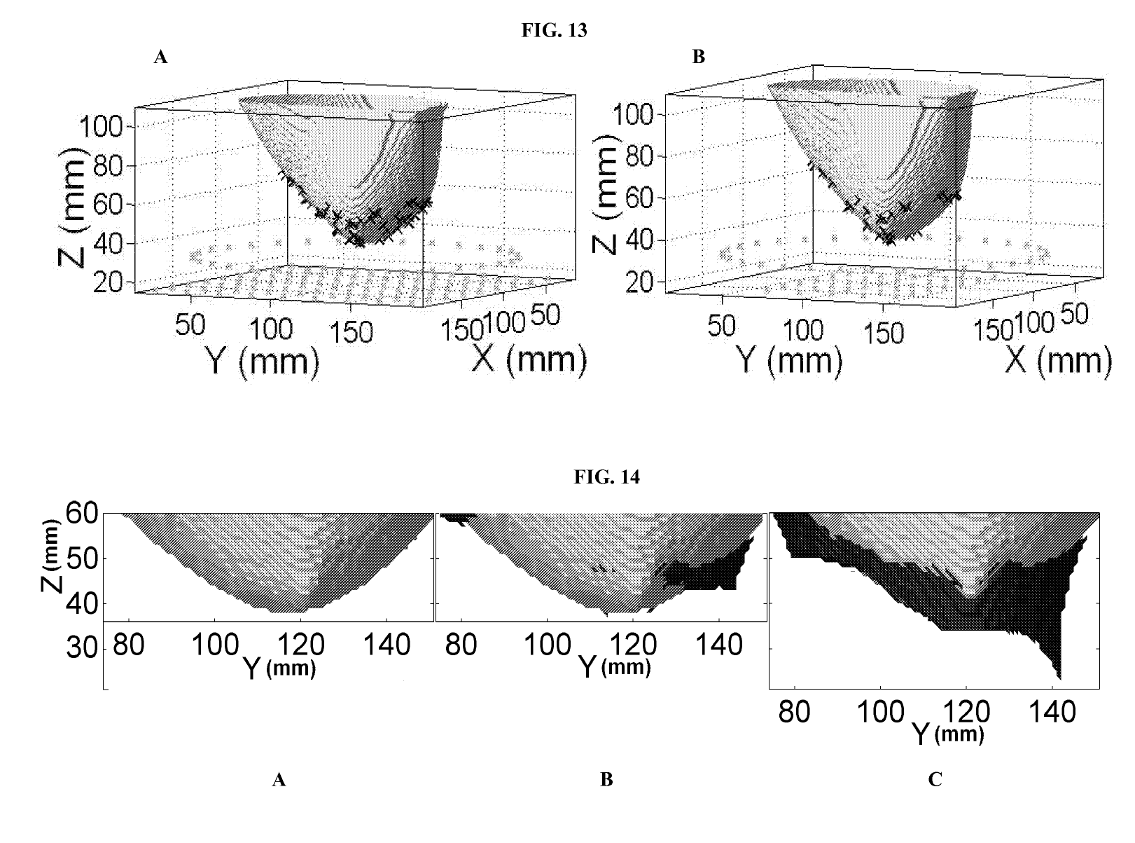

To test the differences in results obtained with various grid scans, the grid size can be systematically reduced, beginning with a 9×9 array scan, which encompasses slightly more than the area of the cage scan. The grid scan patterns can include 7×7, 5×7, 5×5, and 3×3 configurations, for example. One example grid scan pattern has been included for surface outline creation comparison and is a 5×7 grid configuration that is roughly centered in the cage scan (FIG. 10B, FIG. 13B).

FIG. 13 shows results of the estimated and actual antenna locations on the breast for a perimeter row located at z=30 mm with differing grid scans. The “x” indicators in FIG. 13 on the breast show the points on that correspond to closest distance between each antenna and the breast model. The coverage of the breast using a 9×9 grid scan (FIG. 13A) has significantly reduced the surface estimation error. In addition to the nipple location, in this implementation, the large grid has the effect of determining the nipple shape in regions not covered by lower coronal scans. In some embodiments, varying grid sizes may have an effect on breast surface coverage in the nipple region. Analysis of a 5×7 array, for example, as shown in FIG. 13B, indicates several surface areas devoid of antenna location characterization.

Surface outlines for 5×7 and 3×3 grid scans are shown in FIG. 14, and the surface error results for all grid configurations are summarized in Table 2. The simulated breast model is shown in light grey, while the overestimation created by the surface estimate is shown in dark grey. The total surface voxels for the breast outline under z=60 mm was 6908. There are no surface voxels in error greater than 1 mm for the 9×9 grid scan, while for the 5×7 grid scan there are 229 voxels in error greater than 1 mm. The distance error of the largest volume created from actual and estimated volume subtraction for the 5×7 grid scan is 3.2 mm with a mean of 1.6 mm.

For the example shown, the 3×3 grid scan can be adequate for localizing the z-location of the nipple if the pattern is underneath the nipple; the lowest z-location of the overall breast volume, however, can be underestimated due to large areas below z=60 mm not being illuminated. The differences in surface estimation error in this model may not substantially differ between the 5×5 and the 5×7 scan patterns. This may, however, not be the case for another realistic model. In one implementation, the 7×7 grid scan, with very few error sites and 1.4 mm maximum error, may be a superior scan pattern when considering a practical application. The difference of 32 scan positions between a 9×9 grid and a 7×7 grid may decrease the overall scan time and lead to reduced error associated with movement artifacts.

In some cases, the orientation of an antenna can be defined according to certain geometric considerations. The preliminary scan is not limited to a cylindrical scan and also a grid scan in two dimensions. In some cases, the grid scan can move an antenna in three dimensions, for example, an antenna can be scanned in a spherical or hemispherical pattern around a breast. Because the shape and size of human breasts differ among women, a grid scan pattern can be chosen that best accommodates these and other breast characteristics, allowing an optimal initial breast scan to be performed.

### Error Analysis

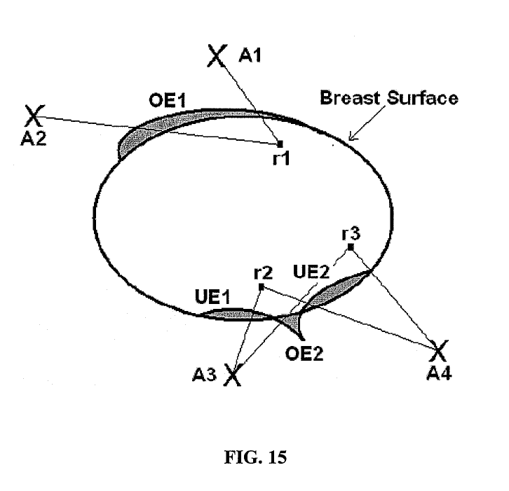

The error associated with the surface estimate can be analyzed and an attempt can be made to quantify the discrepancies between real and estimated breast surfaces placed in a relevant context. In some implementations, the error associated with the surface estimate may lead to errors in antenna placement during the tumor sensing scan, as well as in erroneous wave velocity estimates when focusing to obtain an image. When considering a subsequent antenna scan, an underestimation can be worse than an overestimation, as the antenna may be placed too close to the breast. When considering image focusing, a single erroneous region may mean very little when focusing at one location using a particular antenna, but may have greater impact when focusing at another location using a different antenna, as illustrated in FIG. 15.

FIG. 15 illustrates four areas of erroneous surface estimation, two overestimated (OE1 and OE2) and two underestimated (UE1 and UE2). When considering the antenna positions A1 and A2 and the focus location r1, OE1 can affect the focusing outcome differently for each antenna. Antenna positions A3, A4, and focus locations r2 and r3 illustrate the changing impact of errors as the focus is translated through the imaging region. Finding a relevant quantitative error associated with breast surface discrepancies is, in some cases, not a trivial problem; any single number given to quantify the maximum error associated with a particular method should be qualitatively described and placed in the proper context.

In some cases, the following method can be used to describe the error. Using OE1 in FIG. 15 as a reference, the boundaries of this error region may be considered; specifically the boundary collocated with the breast surface (B1) and the boundary defining the maximum extent of the error region (B2). The minimum distance between a selected pixel on boundary B1 and all pixels on boundary B2 can be determined. The maximum of all of these distances can then be evaluated. The calculated value, in some cases, does not give the maximum dimensions of area or volume in error, instead it attempts to estimate the relevant error associated with focusing. A further discussion of alternative error metrics is provided herein.

Error figures can be directly influenced by the resolution of the imported model and the resolution used in focusing. In one implementation, both resolutions are set to 1 mm to enable direct comparison between simulated and estimated surfaces. Therefore, the minimum error in a region under consideration can be 1 mm. In some implementations, when calculating the average error associated with a specific surface estimate, only erroneous pixels are included. This can create a much larger average error than would be calculated using all points on the estimated breast surface, however, a more relevant average error can be computed. With the current resolution, this can imply that the minimum average error attainable is 1 mm. The number of pixels on the surface can be included and considered erroneous, or Error Sites, used to calculate the average error. This value can be an important gauge as a maximum error value may be identical for different antenna configurations however the quantity of Error Sites may vary. This may offer insight into the relative success of the surface detection algorithm.

### Antenna Placement

### Algorithm

During data collection of a surface estimate, the antenna may be placed at a selected distance from the object being imaged. In some embodiments, the distance may be significant, such as a distance of up to 14 cm. In some embodiments, the surface estimate may be acquired from an alternate imaging modality, like those mentioned above (e.g. audio, RF, optical, infrared). For imaging the interior of the breast, for example, it may be beneficial to locate the antenna close to the breast surface. The results of the surface estimate may be used to place the antennas at a desired distance from the object for a second scan, for example, a high-resolution scan, or a tumor-detection scan if the object is within tissue. An automated algorithm has been created to place the antennas on a row-by-row basis at a desired distance from the surface.

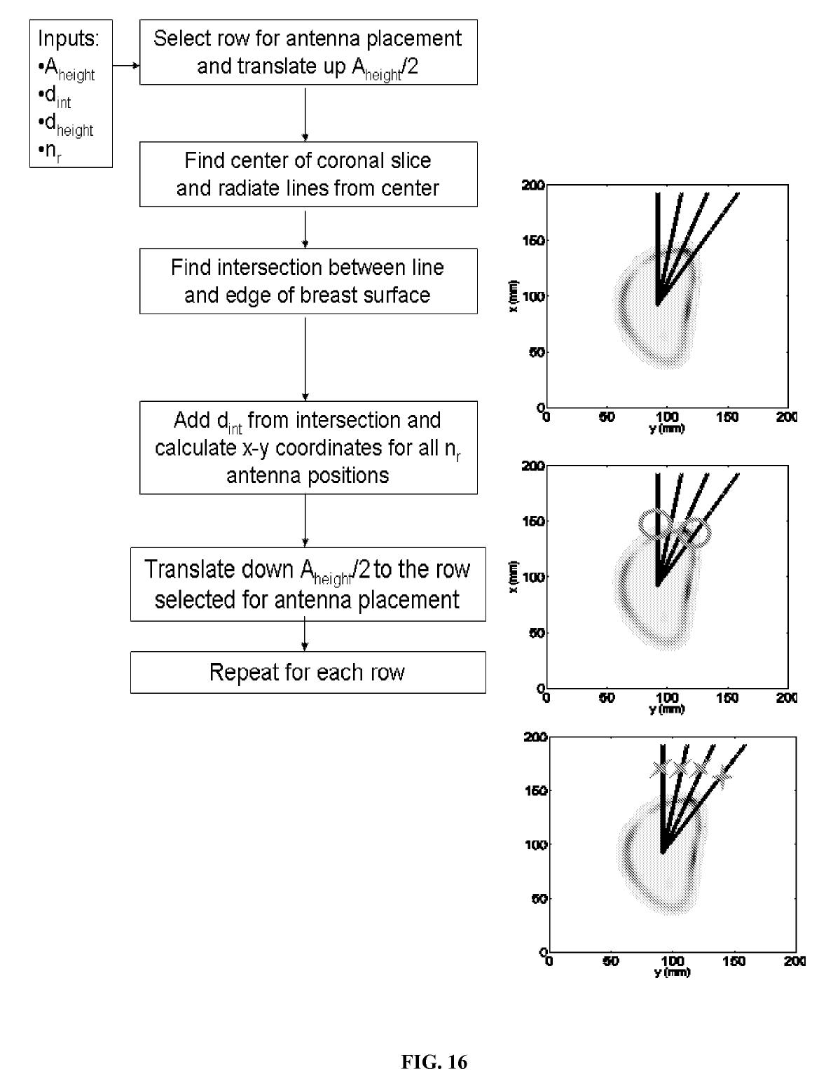

The algorithm is illustrated as a flow diagram in FIG. 16. The desired number of antennas per row (nᵣ), distance from the breast surface (dᵢₙₜ), separation between rows (d_{height}) and antenna height (A_{height}) can be specified by the user. The number of rows of antennas can be dependant on the dimensions of the breast, separation between rows, and antenna height. The dimensions can be estimated with the results of the surface estimate. The first antenna row can be placed with the feed aligned with the estimate of the nipple, and this location is denoted as z1. To determine the separation between the antenna and breast in the x-y plane, the entire antenna structure should be considered. For example, a resistively loaded dipole of length 2 cm can extend 1 cm above and below the feed location.

The location on the breast surface closest to the antenna can be identified, in this case the location closest to the end of the antenna (z=z1+A_{height}/2). Placing the end of the antenna at the desired distance can ensure that the antenna does not touch or couple with the breast; if the feed position is used to determine the separation, the antenna may be placed too close to the breast in areas of curvature such as the chest wall. A coronal slice through the surface estimate can be obtained at the selected z location. The approximate center of the breast surface can be calculated by averaging the maximum and minimum extent of the breast in the x and y directions. A line that radiates out from the estimated center of the breast can be drawn for each desired antenna location.

In one embodiment, the point where the line and edge of the breast surface intersect can be determined using pixel intensities in the surface estimate images. The point can then be translated the desired distance, dᵢₙₜ, from the breast surface along the line. The x-y coordinates can be calculated for this point and translated down A_{height}/2 to the row selected for antenna placement. The process can be repeated for each row required to span the distance between the nipple and chest. The breast surface estimate may also be used to determine the number of antennas per row, as fewer antennas may be required near the nipple. In this case, an estimate of the breast circumference may be obtained from the 2D section through the surface estimate, and divided by the desired separation between antennas in order to determine the number of antennas required in a specific row.

### Example

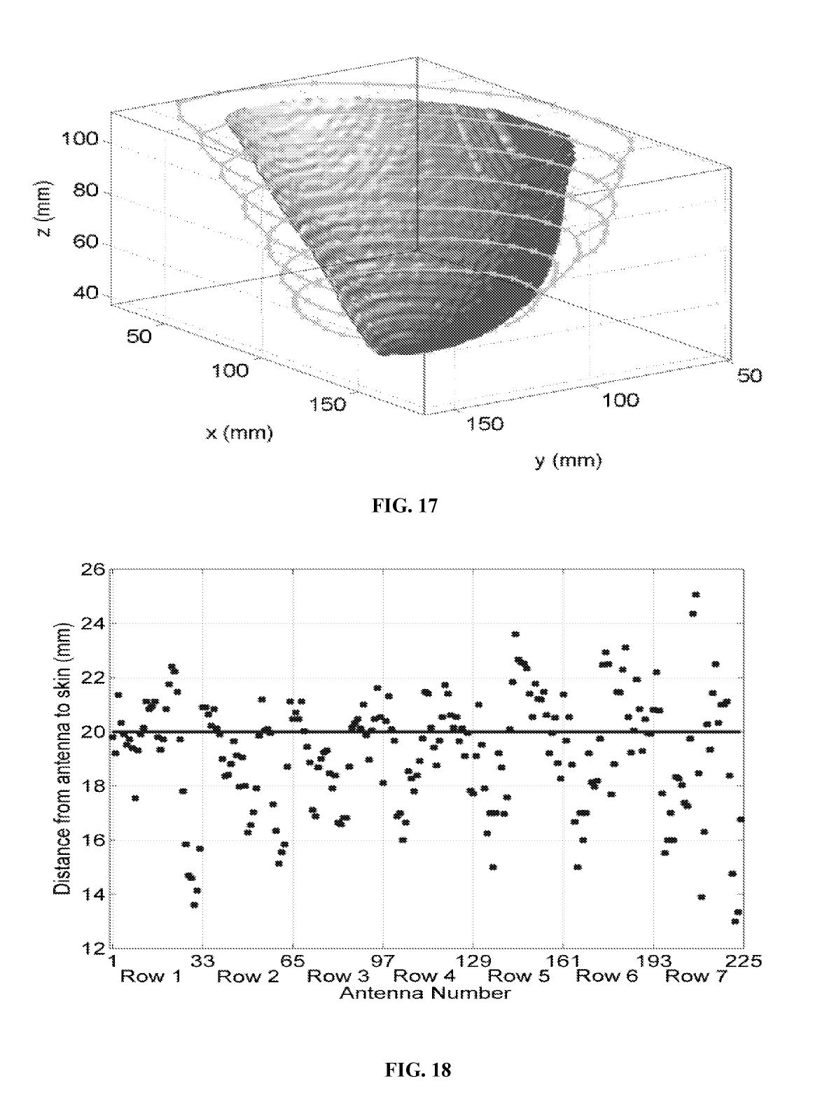

The antenna placement algorithm is applied to the surface estimates created with 9 rows of 32 antennas and breast model 1. For the tumor detection scan, the resistively loaded dipole described above was used and dᵢₙₜ=2 cm, d_{height}=1 cm and A_{height}=2 cm based on antenna dimensions. The resulting antenna positions on breast model 1 are shown in FIG. 17. The distance from antenna feed to the closest skin surface on the actual breast model is plotted in FIG. 18. The average antenna feed to skin distance was found to be 1.92 cm. The variation in distances, as well as the deviation from the 2 cm target can be attributed to the slight underestimation of the breast surface and the complexity of the model. Although the minimum distance from antenna to breast is approximately 1.3 cm in FIG. 18, this is an acceptable placement. Simulations performed with a skin layer at various distances from the resistively loaded dipole antenna demonstrate that the fidelity of the radiated signal and skin reflection is below 0.95 at distances closer than 7.5 mm, so a minimum separation of 7.5 mm may be required.

### Antenna Positioning System

In one general aspect, an antenna positioning system (APS) is provided. In some cases, the positioning system can be used to carry out the scan patterns described herein. In some cases, the APS can be used to scan a breast with an antenna or with multiple antennas. The system can positioned, for example, beneath a patient bed while the patient lies with her breast extending through a hole in the table and extended into a tank that is part of the APS system. The system can scan the antenna around the breast, including from the chest to the nipple and encircling the breast. The pitch of the antenna may change to provide optimal scanning of the breast.

One embodiment of an antenna positioning system 1900 is shown in FIGS. 19A-19D. FIGS. 19A-B illustrates a tank 1910 that can be filled with oil or other immersion medium in which a breast 1920 and antenna 1930 are immersed. An overflow pipe 1950 can capture any oil overflow; for example, if the tank 1910 becomes too full, the oil can flow into a storage reservoir (not shown in FIGS. 19A-19D). The tank 1910 can be connected to a stand 1945. The antenna can be connected to an antenna positioning arm 1965. In some cases, the tank 1910 and stand 1945 can slide on a translation plate 1955. The tank 1910, stand 1945 and translation plate 1955 can rotate on a rotation plate 1960. The breast 1920 does not have to be positioned at the center of the tank 1910. As one embodiment of this system is for microwave breast cancer detection, it may be beneficial that a minimal amount of metal is included in the design. A top-down view of the APS 1900 is shown in FIG. 19C. The antenna 1930 is shown proximal to the breast 1920, and a cable 1923 feeding the antenna 1930 is also indicated. In most cases the cable 1923 can provide energy to drive the antenna 1930.

In general, TSAR reflections may be optimal for imaging purposes when the antenna is located at an optimal distance from the breast in order to record reflections. The selection of the optimal position may be accomplished by translation, rotation, vertical positioning and pitch adjustment of the antenna.

Translation may be used to position the antenna at the correct horizontal distance from the breast. The entire tank 1910 and antenna 1930 can be moved into the desired position using the translation plate 1955. A motor 1968 can be connected to a horizontal threaded rod 1970. When threaded rod 1970 turns, the tank 1910 and stand 1945 can slide along the groove in the translation plate 1955. Vertical positioning of the antenna 1930 can be provided by a motor 1975 connected to a second threaded rod 1976 that is rotatably integrated into the positioning arm 1965 that passes through the stand 1945 (FIG. 19B). Rotation of the threaded rod 1976 can move the positioning arm 1965 and therefore the antenna 1930 in vertical directions (up and down).

Referring now to FIG. 19D, pitch movement of the antenna 1930 can be provided by a rod 1967 that passes through the positioning arm 1965, and couples to a gear 1969 that provides rotation about the longitudinal axis of the rod 1967. The rod 1967 can also be attached to a motor that is included in the positioning arm 1965 (not shown in FIG. 19 as inserted in rod 1965). This gear can be coupled to a gear that is connected to the antenna. When rotated, the gears can change the pitch of the antenna.

In some cases, to perform a scan of the breast, the antenna can be moved to multiple positions encircling the breast. In one embodiment, the entire tank can rotate on the rotation plate in order to provide this function. Before or after rotation, a translation may be required to appropriately position the antenna.

In general, the antenna positioning system can provide flexible scan sequences. In some cases a “row” of data from antenna positions can be collected that encircle the breast. In these cases, the antenna can be positioned near the breast and the system can be rotated to capture data points around the breast circumference. In some cases minor adjustments to the position of the antenna may be warranted.

In some cases the system can provide a scan sequence to collect data in “column” format. In this case, the antenna can scan over a range of vertical positions. Adjustments to positioning (e.g. translation, pitch) may be required during these types of scan.

In some cases, the system can provide scanning of specific points on the surface of the breast or within the volume of the breast. In these cases, the antenna can be moved to specific locations where data are then collected. This type of scanning may be useful, for example, when inspecting a number of suspicious areas rather than scanning the entire breast. Generally, the system can be designed in order to provide flexible scanning capabilities and is extensible to multiple antennas. In some cases, one rotation plate and one translation plate can be incorporated, as in the design shown in FIGS. 19A-19D. In some cases, multiple antenna positioning arms are included, with separate driving motors for each arm. For example, two antennas may be located at opposite positions in the tank. Positioning one antenna may position the second antenna at greater distance from the breast than required for data acquisition. After measurement at the first antenna, it may be required to translate in order to obtain the desired position of the second antenna. One goal of the antenna positioning system may be to reduce data acquisition time, as movement of the antennas can be time consuming. Movement over smaller distances may take less time.

In general, an antenna can be attached to the positioning system by any means. In one embodiment, a cable can connect the antenna to the measurement system by attaching to the antenna and passing through a hole on the antenna positioning arm. This arrangement may minimize cable flex during motion.

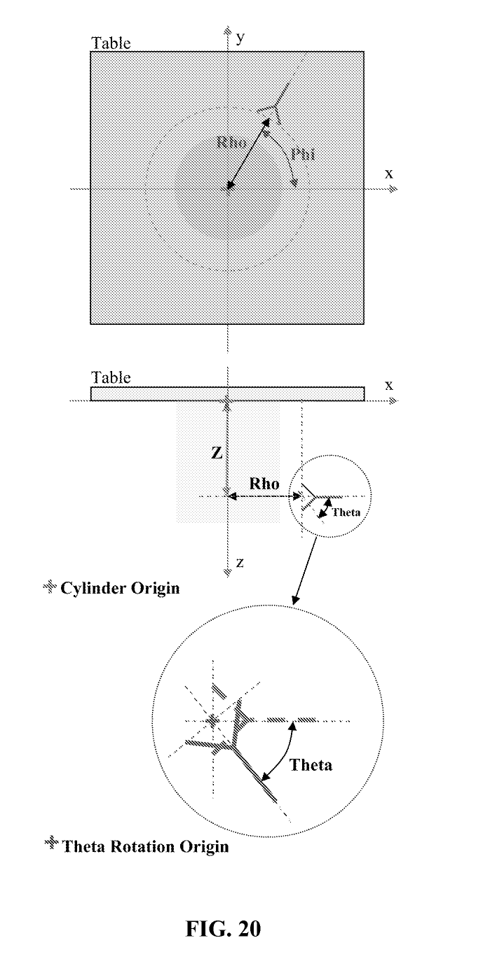

In general, the antenna positioning system can use a cylindrical coordinate system such as that shown in FIG. 20. The origin of the coordinate system can be placed at, for example, the lower center of the table hole. The center of the antenna aperture can be used as reference for the coordinate system. To describe the antenna inclination, a theta component can be added to the antenna aperture as rotation axis.

In general, an antenna fixture system 1980 is provided. In some cases, the fixture system can consist of a plastic plate with a hole to permit access for the antenna cable, two pins to ensure alignment, and two threads to attach the antenna. In some cases, a minimum area of the plate can be dictated by the antenna dimension; its thickness can be variable and chosen to provide optimal functionality for a given arrangement or antenna size. In some cases the connector hole can be located at the center of the plate, and its size can be commensurate with the cable connector diameter.

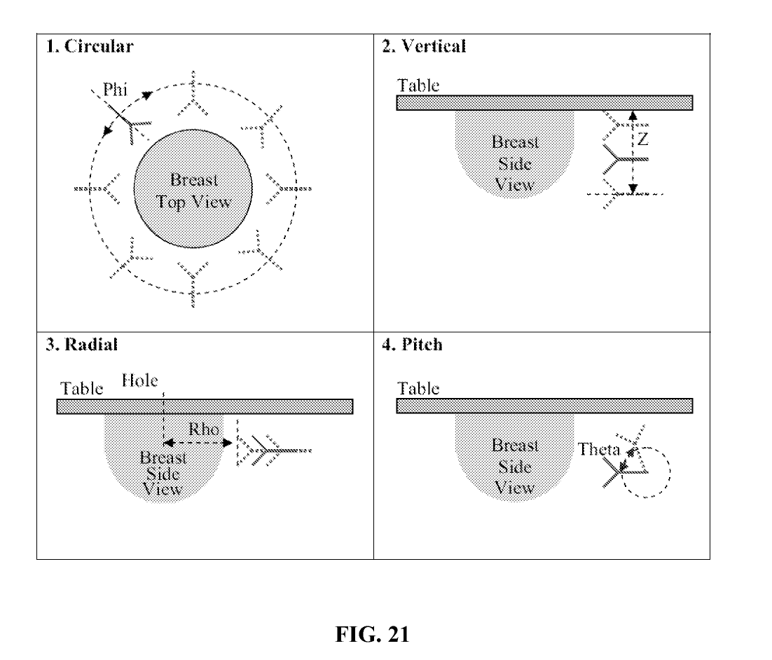

In general, the antenna positioning system can be used to accurately move and place an object (e.g., an antenna) around an object, e.g., a human breast. In general, the antenna positioning system can move in up to as many degrees of freedom as is necessary to suit the particular purpose at hand. In some cases, four degrees of freedom are used: circular movement around the object (Phi), vertical movement, radial movement (Rho), and pitch angle movement (Theta). Exemplary degrees of freedom are illustrated in FIG. 21.

### Other Embodiments

It is to be understood that while the disclosure has been described in conjunction with the detailed description thereof, the foregoing description is intended to illustrate and not limit the scope of the disclosure, which is defined by the scope of the appended claims.

For example, several alternatives to the error analysis described are possible. One approach to error calculation involves determining the error per antenna scan position relative to all focus locations. Calculating the maximum distances that are influenced by over- and underestimation areas gives a number relevant to each antenna and whether its signals are hampered by unacceptably large error. A bar graph may then be generated depicting each antenna and trends may be determined to give insight into the locations of unacceptable over- and underestimation areas. A further improvement would be to determine the error relative to each focus location. A total over- and underestimation distance may be calculated using all included antennas and the distances each signal traveled through erroneous estimations. A surface plot may then be generated of each focused voxel containing this total distance.

Although the methods and variants thereof have been discussed in connection with the Tissue Sensing Adaptive Radar (TSAR) system, they are applicable to other radar-based breast cancer detection systems. The methods and variants thereof are also easily adapted to different antennas.

As described above, alternative embodiments of the first or preliminary scan system 2200 may include a laser 2202, an ultrasound transducer (not shown), and/or a photographic device such as a digital camera 2204. FIG. 22 illustrates one embodiment of a system 2200 that incorporates a plurality of sensors. For example, the system 2200 may include an antenna 1903. The system 2200 may further comprise a laser 2202. In a further embodiment, the system 2200 may include a digital camera 2204. In such an embodiment, each of the antenna 1930, the laser 2202, and the digital camera 2204 may scan the breast tissue 1920. In a further embodiment, the antenna 1930, the laser 2202, and the digital camera 2204 may scan substantially simultaneously.

Additionally, as described in FIG. 22, both the antenna 1930 and the laser 2202 may be coupled to the positioning arm 1965. In such an embodiment, the laser 2202 may facilitate the positioning of the antenna 1930 in preparation for and during the course of the second scan of the breast tissue 1920.

In a further embodiment, the at least one of the antenna 1930, the laser 2202, and/or the digital cameral 2204 may be coupled to a signal processing device 2206. The signal processing device 2206 may comprise a special purpose signal processing device configured with computer readable code. When executed by the signal processing device 2206, the computer readable code may cause the signal processing device to perform operations associated with the various steps and functions of the algorithms described above.

In a further embodiment, the computer readable code may be stored on a tangible computer readable medium 2208. Examples of a tangible computer readable medium 2208 may include a hard disk, a floppy disk, an optical storage disk, a flash memory device, a Random Access Memory (RAM) device, a Read Only Memory (ROM) device such as an EPROM or an EEPROM, and the like. One of ordinary skill in the art will recognize other suitable signal processing devices 2206.

In one embodiment, the signal processing device 2206 may include a Programmable Logic Chip (PLC), a Digital Signal Processor (DSP) device, a Field Programmable Gate Array (FPGA), a microprocessor, a computer processor, or the like. Alternatively, the signal processing device 2206 may comprise a computer suitably programmed to execute the operations of the algorithm described above.

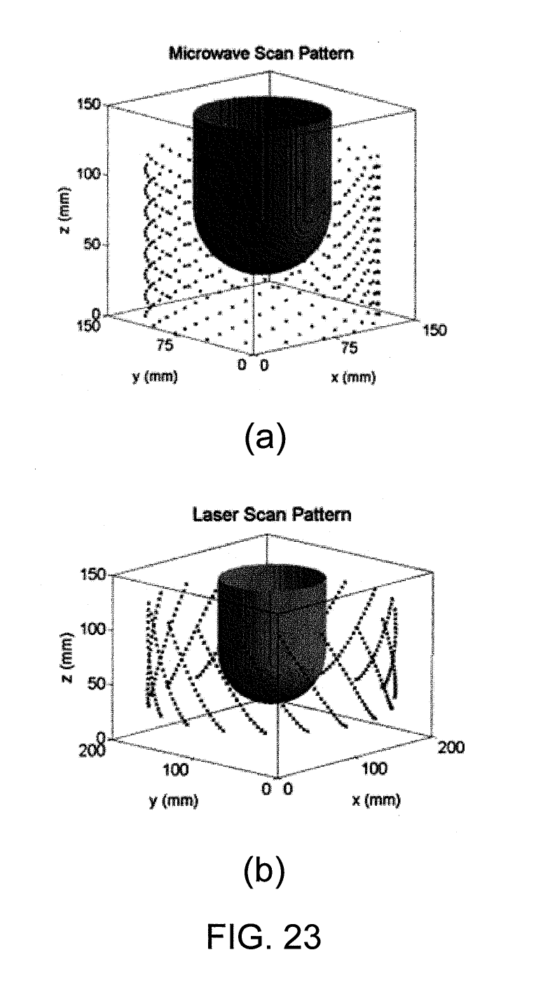

By way of comparison, FIGS. 23A and 23B illustrate one embodiment of a microwave scan pattern and one embodiment of a laser scan pattern. In one embodiment, the microwave scan pattern may include 23 scan rows, with 18 scan points per row. One embodiment of a laser scan pattern may include 21 rows with 18 scan points per row. One of ordinary skill in the art will recognize that alternative scan patterns may be advantageous depending on the application to which the present embodiments are applied.

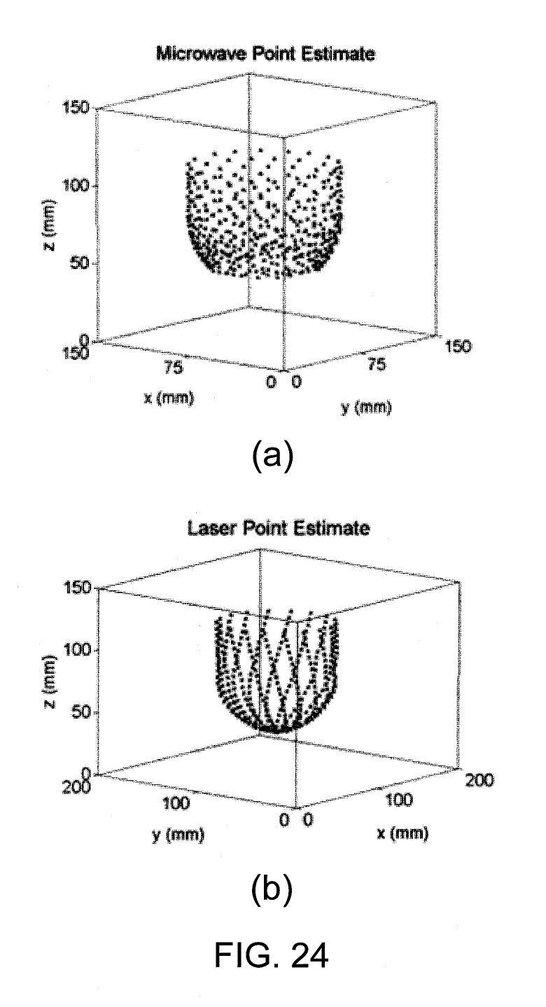

As illustrated in FIGS. 24A and 24B, the laser point estimate may more precisely estimate the surface of the breast in regions of with a higher rate of curvature. For example, the laser point estimate may be substantially more precise than the microwave estimate around the region associated with the nipple. This is demonstrated by the high concentration of laser estimate points around the bottom portion of the diagram.

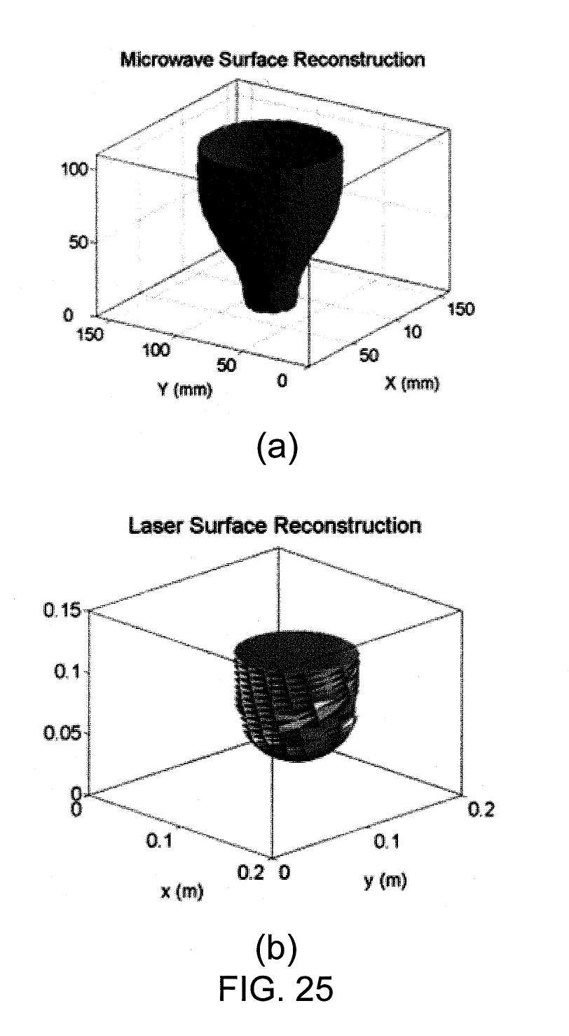

Similarly, the laser surface reconstruction may be more precise than the microwave surface reconstruction, as shown in FIGS. 25A and 25B. In the depicted example, the laser surface reconstruction more precisely models the region around the nipple and more accurately estimates the overall curvature of the entire breast.

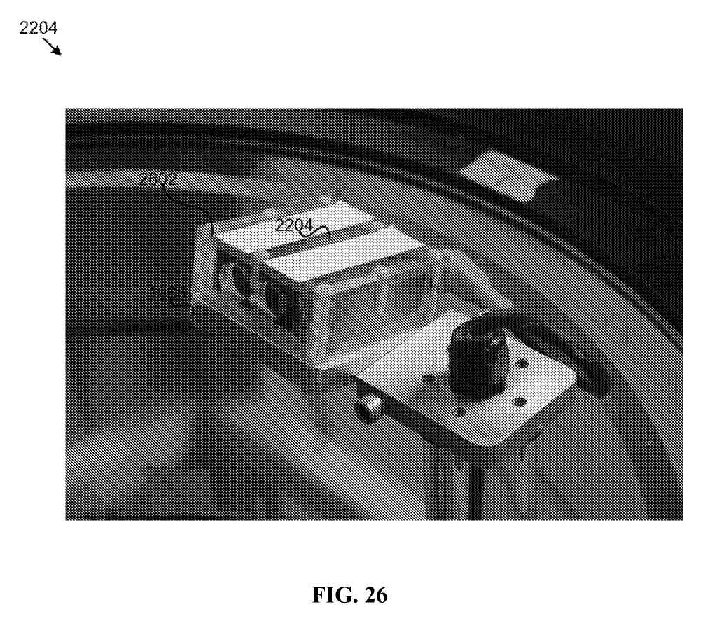

FIG. 26 is a photograph of one embodiment of a laser 2204 that may be implemented in accordance with the present embodiments. In the depicted example, the laser 2204 may be encased in a housing 2602. The laser 2204 may also be coupled to the positioning arm 1965 as illustrated. The positioning arm 1965 may include one or more plates for supporting the laser 2204. In one embodiment, the places may be configured to actuate. In accordance with a predetermined scan pattern.

Other aspects, advantages, and modifications are within the scope of the claims. In light of the described embodiments, one of ordinary skill in the art will recognize methods for adapting, e.g., an ultrasound transducer and/or a digital camera for surface estimation in accordance with the described system and methods.

### Tables

TABLE 1

| | Array | Maximum z | Maximum | Average | Error |
|---|---|---|---|---|---|
| | size | extent (mm) | (mm) | (mm) | Sites |
| | Overestimation Error | | | | |
| Breast Model 1 | 4 × 16 | 100 | 2.24 | 1.18 | 327 |
| | 9 × 16 | 100 | 2.00 | 1.16 | 202 |
| | 9 × 32 | 100 | 2.00 | 1.15 | 230 |
| | 9 × 16 | 110 | 3.61 | 1.29 | 352 |
| | 9 × 32 | 110 | 2.00 | 1.18 | 310 |
| Breast Model 2 | 7 × 16 | 100 | — | — | 0 |
| Breast Model 2B | 7 × 16 | 100 | — | — | 0 |
| | Underestimation Error | | | | |
| Breast Model 1 | 4 × 16 | 100 | 6.16 | 2.02 | 12199 |
| | 9 × 16 | 100 | 6.16 | 1.94 | 9460 |
| | 9 × 32 | 100 | 6.16 | 1.89 | 10644 |
| | 9 × 16 | 110 | 6.16 | 1.93 | 10414 |
| | 9 × 32 | 110 | 6.16 | 1.87 | 11912 |
| Breast Model 2 | 7 × 16 | 100 | 5.74 | 1.98 | 10811 |
| Breast Model 2B | 7 × 16 | 100 | 7.81 | 3.16 | 12927 |

TABLE 2
Summary of surface outline overestimation error using differing grid
scan patterns

| Grid Size | Max Error (mm) | Mean Error (mm) | # of Error Sites |
|---|---|---|---|
| 9 × 9 | 0 | 0 | 0 |
| 7 × 7 | 1.4 | 1.4 | 3 |
| 5 × 7 | 3.2 | 1.6 | 229 |
| 5 × 5 | 3.2 | 1.6 | 232 |
| 3 × 3 | 7.3 | 2.1 | 1275 |

What is claimed is:

1. A method comprising:
receiving, at a sensor, one or more reflections from a surface of an object while the sensor is scanned in a pattern that localizes the surface of the object within a medium;
converting the reflections into one or more signals; and
estimating a position of the surface of the object within the medium in response to the one or more signals.

2. The method of claim 1, further comprising locating a physiological feature on the object using a grid scan, said grid scan comprising:
receiving signals corresponding to reflections from the object surface while the sensor is moved in a pre-defined path; and
determining a distance from the sensor to the physiological feature.

3. The method of claim 1, further comprising performing a scan in a selected area of the surface of the object, wherein an antenna scan pattern is determined in response to information from a coordinate representation of the surface of the object.

4. The method of claim 1, further comprising:
conducting a first scan of the surface of the object with a first sensor; and
conducting a second scan, substantially concurrently with the first scan, of an interior portion of the object with a second sensor.

5. The method of claim 1, further comprising generating a model of the surface of the object in response to a plurality of estimates of the position of a plurality of portions of the surface of the object within the medium.

6. The method of claim 1, further comprising positioning an antenna within a predetermined proximity of the surface of the object in response to the estimate of the position of the surface of the object within the medium.

7. The method of claim 1, further comprising:
emitting a beam of light from a laser directed at the surface of the object;
receiving, at a photo-detector, reflections of the light from the surface of the object; and
converting the received reflections into one or more electrical signals.

8. The method of claim 1, further comprising:
moving an antenna that receives microwave energy reflections from the surface of the object in a pre-selected pattern, the pre-selected pattern being defined to allow overlap of a first geometrically-estimated area calculated from said energy reflection on the object surface at a first antenna location with a second geometrically-estimated area calculated from the energy reflection on the object surface at a second antenna location; and
generating a coordinate representation of the surface of the object.

9. The method of claim 1, further comprising:
emitting an ultrasonic pulse from a transducer, the ultrasonic pulse directed at the surface of the object;
receiving a reflection of the ultrasonic pulse from the surface of the object; and
converting the received reflection into one or more electrical signals.

10. The method of claim 1, further comprising:
capturing a digital image of the surface of the object with a digital camera; and
estimating the location of the surface of the object in response to one or more properties of pixels comprising the digital image.

11. A system comprising:
a sensor configured to:
receive one or more reflections from a surface of an object while being scanned in a pattern that localizes the surface of object within a medium; and
convert the reflections into one or more signals; and
a signal processing device coupled to the sensor, the signal processing device configured to estimate a position of the surface of the object within the medium in response to the one or more signals.

12. The system of claim 11, wherein the signal processing device is further configured to locate a physiological feature on the object in response to data received from a grid scan, the grid scan comprising:
receiving signals corresponding to reflections from the object surface while the sensor is moved in a pre-defined path; and
determining a distance from the sensor to the physiological feature.

13. The system of claim 11, wherein the sensor is further configured to scan in a selected area of the object, wherein a sensor scan pattern is determined in response to information from the coordinate representation or any other means of determining an outline of the surface of the object.

14. The system of claim 11, further comprising:
a first sensor configured to conduct a first scan of the surface of the object; and
a second sensor configured to conduct a second scan, substantially concurrently with the first scan, of an interior portion of the object.

15. The system of claim 11, wherein the signal processing device is further configured to generate a model of the surface of the object in response to a plurality of estimates of the position of a plurality of portions of the surface of the object within the medium.

16. The system of claim 11, further comprising a positioning arm coupled to an antenna, the positioning arm configured to position the antenna within a predetermined proximity of the surface of the object in response to the estimate of the position of the surface of the object within the medium.

17. The system of claim 11, further comprising:
a laser configured to emit a beam of light directed at the object; and
a photo-detector configured to:
receive reflections of the light from the surface of the object; and
convert the received reflections into one or more electrical signals.

18. The system of claim 11, further comprising:
an antenna configured to receive microwave energy reflections from an object surface; and
a positioning arm coupled to the antenna, the positioning arm configured to move the antenna in a pre-selected pattern, the pre-selected pattern being defined to allow overlap of a first geometrically-estimated area calculated from the energy reflection on the object surface at a first antenna location with a second geometrically-estimated area calculated from the energy reflection on the object surface at a second antenna location.

19. The system of claim 11, further comprising:
an ultrasound transducer configured to:
emit an ultrasonic pulse directed at the surface of the object;
receive a reflection of the ultrasonic pulse from the surface of the object; and
convert the received reflection into one or more electrical signals.

20. The system of claim 11, further comprising:
a digital camera configured to capture a digital image of the surface of the object; and
the signal processing device coupled to the digital camera, the signal processing device configured to estimate the location of the surface of the object in response to one or more properties of pixels comprising the digital image.
